(19) United States
(12) Patent Application Publication
(10) Pub. No.: US 2026/0348984 A1
(43) Pub. Date: Nov. 5, 2026

(54) Method of Diagnosis and Treatment of Alzheimer's Disease

(71) Applicant: Molecular You Corporation, Vancouver (CA)

(72) Inventors: Mohammad Ashraful Anwar, Calgary (CA); Zhengnan Wang, Vancouver (CA); Ana Maddox, County of Two Hills (CA); Ammar Hassanzadeh Keshteli, Edmonton (CA); Robert Fraser, Vancouver (CA)

(21) Appl. No.: 19/486,938

(22) PCT Filed: May 24, 2024
(86) PCT No.: PCT/CA2024/050694

§ 371 Date: Nov. 23, 2025

Related U.S. Application Data
(60) Provisional application No. 63/504,341, filed on May 25, 2023.

(51) Int. Cl. G01N 33/68; G16H 10/40

(52) U.S. Cl. CPC G01N 33/6896; G16H 10/40; G01N 2570/00; G01N 2800/2821; G01N 2800/52; G01N 2800/56

(57) ABSTRACT

Provided herein is a method for diagnosing and treating Alzheimer's disease comprising: (a) providing a biological sample obtained from the subject; (b) measuring concentration levels from the obtained sample, at least one, at least two, at least three, at least four or at least five Alzheimer's-related metabolites described herein and/or at least one, at least two, at least three, at least four or at least five Alzheimer's-related proteins described herein; (c) comparing the concentration levels of the Alzheimer's-related metabolites and/or proteins from the obtained sample to the concentration levels of corresponding reference Alzheimer's-related metabolites and/or proteins from an Alzheimer's-negative sample; (d) identifying the subject as having Alzheimer's if the concentration levels of the Alzheimer's-related metabolites and/or proteins from the obtained sample are different relative to the concentration levels of the reference Alzheimer's-related metabolites and/or proteins from the Alzheimer's-negative sample; and (e) treating or causing treatment of the subject.

### TECHNICAL FIELD

The present disclosure relates generally to the field of proteomic and/or metabolomic assessment of subjects with Alzheimer's disease.

### BACKGROUND

Alzheimer's disease is a progressive neurodegenerative disorder characterized by the loss of memory and cognitive abilities. The disease is caused by the accumulation of amyloid plaques and neurofibrillary tangles in the brain. These plaques and tangles disrupt communication between brain cells and trigger inflammation, leading to the death of neurons and the shrinkage of brain tissue. Additionally, the disease is associated with a reduction in neurotransmitters, particularly acetylcholine, which are critical for normal brain function. As a result, Alzheimer's disease gradually impairs memory, thinking, language, judgment, and/or movement.

The amyloid plaques are formed when abnormal deposits of a protein called beta-amyloid build up in the spaces between nerve cells in the brain. These plaques cause the inflammation, leading to the death of neurons and further plaque formation. The accumulation of amyloid plaques also triggers the formation of the tangles, which are abnormal aggregates of another protein called tau.

The tangles disrupt the transportation of nutrients and other essential substances within brain cells, leading to the death of neurons and the decline in cognitive function seen in Alzheimer's disease. The combination of the amyloid plaques and the neurofibrillary tangles cause widespread damage to brain tissue, leading to the progressive decline in cognitive function that characterizes Alzheimer's disease.

It is desirable to obtain an accurate diagnosis of Alzheimer's disease. The correct diagnosis is an important first step towards receiving the appropriate treatment, care, family education and planning for the future. Early signs of Alzheimer's disease include minor memory issues, such as forgetting new information and having difficulty completing familiar tasks. Early-stage Alzheimer's disease can be difficult to diagnose, especially since cognitive impairment can be difficult to measure. Furthermore, it can be difficult to rule out other disorders that cause similar symptoms, such as thyroid disorders and vitamin B-12 deficiency. Currently, physicians use mental status tests and evaluate the scores from such tests to determine the degree of cognitive impairment. While researchers are examining more accurate ways to diagnose Alzheimer's disease (including assessing biomarkers such as tau), progress in the field has been slow.

Accordingly, an improved method is needed that can offer a more reliable diagnosis of Alzheimer's disease, the risk of developing the disease, and treatment thereof. Such a method may further provide improved methods of monitoring disease progression.

### SUMMARY

The present disclosure provides a method for diagnosing Alzheimer's disease, or assessing a risk of developing the disease, and treating or causing the treating thereof. Additionally or alternatively, such method may further provide improved methods of monitoring Alzheimer's disease progression.

According to the present disclosure, a new metabolic and/or proteomic profile for Alzheimer's disease is used to diagnose, assess a risk of developing the disease, manage symptoms, monitor disease progression and/or ameliorate the disease.

According to the present disclosure, a unique combination of metabolomic and/or proteomic signatures are identified that have high predictive value for Alzheimer's disease and significantly improve the identification of and treatment of the disease relative to previous methods. In one non-limiting example, at least one, at least two, at least three or all metabolites selected from Trigonelline, Lysophosphatidylcholine (lysoPC) (including but not limited to C18:2 fatty acid metabolite, and/or C6:1 fatty acid metabolite), Hydroxyphenyl acetic acid (HPA) and homocysteine and/or at least one, at least two, at least three, at least four, at least five, at least six, at least seven, at least eight or all Alzheimer's-related proteins or fragments thereof selected from Coagulation Factor XIII A chain, Complement factor D, C-reactive protein, IgGFc-binding protein, Ceruloplasmin, Haptoglobin, Tenascin C and/or Serum Amyloid A-1 are used to identify a subject with Alzheimer's risk or having the disease via a metabolomic/proteomic analysis carried out on a biological sample. After identifying the subject as having Alzheimer's disease or being at risk of developing the disease, appropriate regimes to treat, prevent and/or ameliorate the disease are carried out.

In additional or alternative embodiments, the new metabolic and/or proteomic profile is used to monitor disease progression or provide improvements in the disease condition.

In one embodiment, the new metabolite and/or proteomic profile for Alzheimer's disease comprises at least one, at least two, at least three, at least four, at least five, at least six or at least seven Alzheimer's-related metabolites selected from Hydroxyphenylacetic acid (HPA); Trigonelline; a Lysophosphatidylcholine (lysoPC), e.g., selected from a C18:2, C18:0, C18:1, C16:0, C14:0, C16:1, C20:4, C26:0 or C17:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin; and/or at least one, at least two, at least three, at least four, at least five or at least six Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

According to an aspect of the disclosure, there is provided a method for diagnosing and treating Alzheimer's disease in a subject, the method comprising: (a) providing a biological sample obtained from the subject; (b) measuring concentration levels from the obtain sample, at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0 or C18:2 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor; (c) comparing the concentration levels of the Alzheimer's-related metabolites and/or Alzheimer's-related proteins from the obtained sample to the concentration levels of corresponding reference Alzheimer's-related metabolites and/or Alzheimer's-related proteins from an Alzheimer's-negative sample; (d) identifying the subject as having Alzheimer's disease if the concentration levels of the Alzheimer's-related metabolites and/or Alzheimer's-related proteins from the obtained sample are different relative to the concentration levels of the reference Alzheimer's-related metabolites and/or Alzheimer's-related proteins from the Alzheimer's-negative sample; and (e) optionally treating or causing treatment of the subject so identified with an Alzheimer's treatment regime.

In one embodiment of any aspect of the disclosure, the Alzheimer's-related metabolites are selected from at least Hydroxyphenylacetic acid (HPA); Trigonelline; Lysophosphatidylcholine (lysoPC); C18:2 fatty acid metabolite; C6:1 fatty acid metabolite; and/or at least Homocysteine; and the Alzheimer's-related proteins are selected from Coagulation factor XIII A Chain; Complement factor D; IgGFc-binding protein; Serum amyloid A-1 protein; Ceruloplasmin; Haptoglobin; C-reactive protein and/or Tenascin C.

In one embodiment of any aspect of the disclosure, the Alzheimer's-related metabolites are selected from at least Hydroxyphenylacetic acid; or Trigonelline; and the Alzheimer's-related proteins are selected from at least Coagulation factor XIII A Chain; Complement factor D; or C-reactive protein.

According to another aspect of the disclosure, there is provided a method for diagnosing and optionally treating Alzheimer's disease in a subject, the method comprising: (a) providing a biological sample obtained from the subject; (b) measuring from the obtain sample or having measured in a spectroscopy unit the concentration levels of at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin, Kallistatin; Xaa-Pro dipeptide; Lipopolysaccharide-binding protein; Insulin-like growth factor; and/or Von Willebrand factor; (c) comparing or having compared concentration levels of the Alzheimer's-related metabolites and/or the Alzheimer's-related proteins as determined in the spectroscopy unit to the concentration levels of reference Alzheimer's-related metabolites and/or Alzheimer's-related proteins from an Alzheimer's-negative sample; (d) identifying the subject as having Alzheimer's if the concentration levels of the Alzheimer's-related metabolites and/or the Alzheimer's-related proteins from the obtained sample are different relative to the concentration levels of the reference Alzheimer's-related metabolites and/or Alzheimer's-related proteins from the Alzheimer's-negative sample; and (e) optionally treating or causing treatment of the subject so identified with an Alzheimer's treatment regime.

In one embodiment of the foregoing aspect of the disclosure, the Alzheimer's-related metabolites are selected from at least Hydroxyphenylacetic acid (HPA); Trigonelline; Lysophosphatidyl choline (lysoPC); C18:2 fatty acid metabolite; C6:1 fatty acid metabolite; and/or at least Homocysteine; and the proteins are selected from Coagulation factor XIII A Chain; Complement factor D; IgGFc-binding protein; Serum amyloid A-1 protein; Ceruloplasmin; C-reactive protein; Tenascin C; Serum amyloid A-1 and/or Haptoglobin.

In one embodiment of the foregoing aspect of the disclosure, the Alzheimer's-related metabolites are selected from at least Hydroxyphenylacetic acid (HPA); or Trigonelline; and the Alzheimer's-related proteins are selected from Coagulation factor XIII A Chain or Complement factor D.

In one embodiment of any aspect of the disclosure, the optional Alzheimer's treatment regime comprises adjusting the blood levels of one or more of the Alzheimer's-related metabolites and/or the Alzheimer's-related proteins in the subject diagnosed as having the Alzheimer's or predisposed of developing the Alzheimer's or a combination thereof.

In one embodiment of any aspect of the disclosure, the adjustment of the blood levels of one or more of the Alzheimer's-related metabolites and/or the Alzheimer's-related proteins in the subject occurs until an improvement in the Alzheimer's disease symptoms in the subject is observed.

In one embodiment of any aspect of the disclosure, wherein the adjustment of the blood levels of one or more of the Alzheimer's-related metabolites comprises adjusting the composition of gut microbiota in the subject.

In one embodiment of any aspect of the disclosure, the identifying occurs upon determination that the concentration levels of at least one, at least two, at least three, at least four or at least five of the Alzheimer's-related metabolites and/or the Alzheimer's-related proteins from the obtained sample differ by about 20% or more, about 30% or more, about 40% or more, about 50% or more, about 60% or more, or about 70% or more relative to the concentration levels of the reference Alzheimer's-related proteins from the Alzheimer's-negative sample.

In one embodiment of any aspect of the disclosure, the identifying occurs upon determination that the concentration levels of the Homocysteine, C18:2 fatty acid metabolite and/or C6:1 fatty acid metabolite from the obtained sample are decreased relative to the concentration levels of the corresponding reference Alzheimer's-related metabolites and/or Alzheimer's-related proteins from the Alzheimer's-negative sample.

In one embodiment of any aspect of the disclosure, the identifying occurs upon determination that the concentration level of Coagulation factor XIII A Chain and/or Hydroxyphenylacetic acid (HPA) from the obtained sample are increased relative to the concentration levels of the corresponding reference Alzheimer's-related metabolites and/or Alzheimer's-related proteins from the Alzheimer's-negative sample.

In one embodiment of any aspect of the disclosure, the obtained sample is blood or urine, preferably serum, plasma or urine.

In one embodiment of any aspect of the disclosure, the Alzheimer's-related metabolites and/or the Alzheimer's-related proteins are measured by a spectroscopic technique, wherein the spectroscopic technique is selected from the group consisting of liquid chromatography, gas chromatography, liquid chromatography mass spectrometry, gas chromatography mass spectrometry, high performance liquid chromatography mass spectrometry, capillary electrophoresis mass spectrometry, nuclear magnetic resonance spectrometry (NMR), raman spectroscopy, and infrared spectroscopy.

In one embodiment of any aspect of the disclosure, the comparison of the concentration levels of the Alzheimer's-related metabolites and/or the Alzheimer's-related proteins from the obtained sample to the concentration levels of the reference Alzheimer's-related metabolites and/or Alzheimer's-related proteins from the Alzheimer's-negative sample comprises using multivariate statistical analysis.

In one embodiment of any aspect of the disclosure, the multivariate statistical analysis is selected from principal component analysis (PCA), or partial least squares projects to latent structures discriminant analysis (PLS-DA).

According to another aspect of the disclosure, there is provided a method of monitoring Alzheimer's disease progression thereof and optionally treating the Alzheimer's disease in a subject, the method comprising: (a) providing a first biological sample obtained from the subject at a first time; (b) assessing a first Alzheimer's-related metabolite and/or Alzheimer's-related proteomic profile by measuring concentration levels of at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor from the first obtained sample; (c) comparing the first Alzheimer's-related metabolite and/or Alzheimer's-related proteomic profile with a reference Alzheimer's-related metabolite profile and/or Alzheimer's-related proteomic profile from an Alzheimer's-negative sample; (d) determining that there is a first difference between the first Alzheimer's-related metabolite and/or Alzheimer's-related proteomic profile and the reference Alzheimer's-related metabolite and/or Alzheimer's-related proteomic profile from the Alzheimer's-negative sample, the first difference being indicative of Alzheimer's; (e) providing a second biological sample obtained from the subject at a second time that is after the first time; (f) assessing a second Alzheimer's-related metabolite and/or Alzheimer's-related proteomic profile by measuring concentration levels of the Alzheimer's-related metabolites and/or Alzheimer's-related proteins from the second obtained sample; (g) comparing the second Alzheimer's-related metabolite and/or Alzheimer's-related proteomic profile with the reference Alzheimer's-related metabolite and/or proteomic profile from the Alzheimer's-negative sample; (h) determining that there is a second difference between the first Alzheimer's-related metabolite and/or proteomic profile and the reference Alzheimer's-related metabolite and/or proteomic profile from the Alzheimer's-negative sample, the second difference being indicative of Alzheimer's; (i) determining Alzheimer's progression based on at least in part on the first and second differences; and (j) optionally treating the subject as identified with an Alzheimer's treatment regime.

According to another aspect of the disclosure, there is provided a kit for use in any embodiment or aspect of the disclosure, comprising reagents for measuring concentration levels of the Alzheimer's-related metabolites and/or the Alzheimer's-related proteins, optionally together with instructions for use.

According to another aspect of the disclosure, there is provided a kit for diagnosis Alzheimer's disease comprising: (a) a detector configured to detect concentration levels of at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor from an obtained biological sample; (b) a composition comprising the corresponding Alzheimer's-related metabolites and/or Alzheimer's-related proteins in control levels corresponding to a control group of Alzheimer's-negative subjects; (c) a multivariate analysis system configured to analyze a difference in the concentration levels of the Alzheimer's-related metabolites and/or Alzheimer's-related proteins and the control levels, and (d) optionally, instructions for an Alzheimer's diagnosis method; wherein the method comprises measuring, using the detector, the levels of the Alzheimer's-related metabolites and/or Alzheimer's-related proteins from the obtained biological sample, and comparing the levels of the obtained Alzheimer's-related metabolites and/or Alzheimer's-related proteins to the control levels of the Alzheimer's-related metabolites and/or Alzheimer's-related proteins obtained from Alzheimer's-negative subjects.

In one embodiment of the foregoing aspect of the disclosure, the Alzheimer's-related metabolites are selected from at least Hydroxyphenylacetic acid (HPA); Trigonelline; Lysophosphatidyl choline; C18:2 fatty acid metabolite; C6:1 fatty acid metabolite; and/or at least Homocysteine; and the Alzheimer's-related proteins are selected from at least Coagulation factor XIII A Chain; Complement factor D; IgGFc-binding protein; Serum amyloid A-1 protein; Ceruloplasmin; C-reactive protein; Tenascin C; Serum amyloid A-1 and/or Haptoglobin.

In one embodiment of the foregoing aspect of the disclosure, the Alzheimer's-related metabolites are selected from at least Hydroxyphenylacetic acid (HPA); or Trigonelline; and the Alzheimer's-related proteins are selected from Coagulation factor XIII A Chain, C-reactive protein, Tenascin C, Serum amyloid A-1 or Complement factor D.

In one embodiment of the foregoing aspect of the disclosure, the detector comprises a multi-metabolite detector and/or multi-proteomic detector configured to measure the levels of the Alzheimer's-related metabolites and/or the Alzheimer's-related proteins.

According to another aspect of the disclosure, there is provided a computer-implemented method for processing a biological sample of a subject, diagnosing Alzheimer's disease and optionally causing treating of the Alzheimer's, the computer-implemented method comprising: (a) receiving a biological sample obtained from the subject; (b) processing the sample in a spectroscopy unit directly or wirelessly linked to a processing device, the processing device having memory for storing measurement data from the spectroscopy unit; (c) in the spectroscopy unit, measuring levels of at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0; C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor and storing the measurement data in the processor; or (d) comparing the stored measurement data to a value in the memory representing an Alzheimer's-negative sample using multivariate statistical analysis; and (e) storing on the processing device a result corresponding to at least one, at least two, at least three, at least four or at least five Alzheimer's-related metabolites and/or Alzheimer's-related proteins from the obtained sample, wherein the result identifies the subject as having Alzheimer's if the measurement data representing the levels of the Alzheimer's-related metabolites and/or Alzheimer's-related proteins are different relative to a concentration levels of reference Alzheimer's-related metabolites and/or Alzheimer's-related proteins from an Alzheimer's-negative sample; (f) displaying an Alzheimer's treatment regime on an electronic display connected directly or wirelessly to the processor for the subject identified as having Alzheimer's or as having predisposition of developing Alzheimer's, the displayed treatment regime comprising electronic text on a graphical user interface; and (iv) optionally causing the adjusting of blood levels of one or more of the Alzheimer's-related metabolites and/or Alzheimer's-related proteins in the subject diagnosed as having or predisposed of developing the Alzheimer's until an improvement in the cognitive performance in the subject is observed.

According to the foregoing aspect of the disclosure, the Alzheimer's-related metabolites are selected from Hydroxyphenylacetic acid (HPA); Trigonelline; Lysophosphatidyl choline; C18:2 fatty acid metabolite; C6:1 fatty acid metabolite; and/or at least Homocysteine; and the proteins are selected from at least Coagulation factor XIII A Chain; Complement factor D; IgGFc-binding protein; Serum amyloid A-1 protein; Ceruloplasmin; C-reactive protein, Tenascine C and/or Haptoglobin.

According to the foregoing aspect of the disclosure, the Alzheimer's-related metabolites are selected from at least Hydroxyphenylacetic acid (HPA); or Trigonelline; and the Alzheimer's-related proteins are selected from at least Coagulation factor XIII A Chain or Complement factor D.

According to a further aspect of the disclosure, there is provided a method for diagnosing and optionally treating Alzheimer's disease comprising: (a) obtaining a signature of metabolites and proteins from a biological sample of a subject, the signature obtained by measuring Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor; (b) classifying the patient into an Alzheimer's disease risk class or healthy class using a statistical analysis that has at least 85% predictive value comprising comparing the signature so obtained with a control signature, the classifying comprising a computer-implemented statistical analysis; (c) identifying the subject as having Alzheimer's disease or a risk of development thereof if the subject is classified into the Alzheimer's disease risk class; and (d) optionally treating or causing the treatment of the subject so identified in step (c) with a drug effective to treat, ameliorate or reduce the symptoms Alzheimer's disease, wherein the treating optionally comprises adjusting the levels of one or more of the metabolites and/or proteins identified in the signature as being present at levels different than the control.

According to embodiments of the foregoing aspect of the disclosure, the Alzheimer's-related metabolites are selected from at least Hydroxyphenylacetic acid (HPA); Trigonelline; Lysophosphatidyl choline; C18:2 fatty acid metabolite; C6:1 fatty acid metabolite; and/or at least Homocysteine; and the Alzheimer's-related proteins are selected from at least Coagulation factor XIII A Chain; Complement factor D; IgGFc-binding protein; Serum amyloid A-1 protein; Ceruloplasmin; C-reactive protein; Tenascin C; Serum amyloid A-1 and/or Haptoglobin.

According to embodiments of the foregoing aspect of the disclosure, the Alzheimer's-related metabolites are selected from at least Hydroxyphenylacetic acid (HPA); or Trigonelline; and the Alzheimer's-related proteins are selected from at least Coagulation factor XIII A Chain, Complement factor D or C-reactive protein.

According to embodiments of any of the foregoing aspects of the disclosure, the method further comprises adjusting or causing the adjustment of the levels of the metabolites and/or proteins comprises nucleic acid therapy. In one embodiment, the nucleic acid therapy comprises reducing the level of a protein expressed or metabolite produced by siRNA or antisense therapy.

According to embodiments of any of the foregoing aspects of the disclosure, the nucleic acid therapy comprises increasing the level of a protein expressed or metabolite produced by mRNA therapy.

According to embodiments of any of the foregoing aspects of the disclosure, the metabolite and/or proteome profile measured is based on a profile identified in a previous computer-implemented statistical analysis model that has a predictive value of at least 90% and comprises at least one of means comparison, PCA, PLS-DA or recursive SVM data analyses.

According to embodiments of any of the foregoing aspects of the disclosure, the metabolite and/or proteome profile has been previously identified as having the predictive value by classifying disease and non-disease samples into the two groups by a computer-implemented method based on the predicted metabolites and/or proteins used in the computer model and assessing whether the groups are separated.

According to another aspect of the disclosure, there is provided a method of identifying a subject's risk of developing Alzheimer's disease or identifying the subject as having Alzheimer's disease comprising: (a) obtaining one or more biological samples from the subject; (b) conducting a metabolomic and proteomic analysis of the one or more biological samples; (c) measuring at least at least one, at least two, at least three or all metabolites selected from Trigonelline, lysophosphatidylcholine (e.g., C18:2 fatty acid metabolite and/or C6:1 fatty acid metabolite) and/or hydroxyphenyl acetic acid (HPA) and at least one, at least two, at least three or all proteins or fragments thereof selected from coagulation factor XIIIA chain, Complement factor D, C-reactive protein, IgGFc-binding protein, Ceruloplasmin, Haptoglobin, Tenascin C and/or Serum Amyloid A-1, optionally wherein the metabolite(s) and/or protein(s) measured have a predictive score of at least 0.5 as determined on a VIP plot; (d) identifying the subject as having Alzheimer's disease or a risk of development thereof if the levels of the metabolite(s) and/or protein(s) differ from a control reference; and (e) optionally providing results of the metabolomic and/or proteomic analysis or a treatment regime based on said analysis via an on-line platform if the subject is identified as having Alzheimer's disease or at risk of development thereof.

According to any one of the foregoing aspects or embodiments thereof, the proteins or fragments thereof and/or metabolites are at least one, two or all of Homocysteine, C18:2 fatty acid metabolite and C6:1 fatty acid metabolite and wherein measured levels of the corresponding at least one, two or all of Homocysteine, C18:2 fatty acid metabolite and C6:1 fatty acid metabolite are reduced relative to an Alzheimer's-negative sample.

According to any one of the foregoing aspects or embodiments thereof, the proteins, fragments thereof and/or metabolites comprise at least Homocysteine and C18:2 fatty acid metabolite.

According to any one of the foregoing aspects or embodiments thereof, the proteins, fragments thereof and/or metabolites comprise at least Homocysteine and C6:1 fatty acid metabolite.

According to any one of the foregoing aspects or embodiments thereof, the proteins, fragments thereof and/or metabolites comprise at least C18:2 fatty acid metabolite and C6:1 fatty acid metabolite.

### BRIEF DESCRIPTION OF THE DRAWINGS

While the specification concludes with claims particularly pointing out and distinctly claiming the disclosure, it is believed that the disclosure will be better understood from the following description of the accompanying figures wherein:

FIG. 1A shows the age range of the cohort.

FIG. 1B shows the number of males and females in the cohort.

FIG. 1C shows the age distribution by sex of the cohort.

FIG. 2A shows statistical metabolic biomarker data for lysophosphatidylcholine (lysoPC) C18:2. The left plot shows the original concentration and the left plot normalized concentration data.

FIG. 2B shows statistical metabolic biomarker data for lysoPC C18:0. The left plot shows the original concentration and the left plot normalized concentration data.

FIG. 2C shows statistical metabolic biomarker data for lysoPC C18:1. The left plot shows the original concentration and the left plot normalized concentration data.

FIG. 2D shows statistical metabolic biomarker data for lysoPC C16:0. The left plot shows the original concentration and the left plot normalized concentration data.

FIG. 2E shows statistical metabolic biomarker data for lysoPC C14:0. The left plot shows the original concentration and the left plot normalized concentration data.

FIG. 2F shows statistical metabolic biomarker data for lysophosphatidylcholine (lysoPC) C16:1. The left plot shows the original concentration and the left plot normalized concentration data.

FIG. 2G shows statistical metabolic biomarker data for Trigonelline. The left plot shows the original concentration and the left plot normalized concentration data.

FIG. 2H shows statistical metabolic biomarker data for lysophosphatidylcholine (lysoPC) C20:4. The left plot shows the original concentration and the left plot normalized concentration data.

FIG. 2I shows statistical metabolic biomarker data for lysophosphatidylcholine (lysoPC) C17:0. The left plot shows the original concentration and the left plot normalized concentration data.

FIG. 2J shows statistical metabolic biomarker data for Citrulline. The left plot shows the original concentration and the left plot normalized concentration data.

FIG. 3 is a principal components analysis (PCA) scatter plot of the metabolites tested in Example 1.

FIG. 4 is a 2D PCA scores plot of the metabolites tested in Example 1.

FIG. 5 is a partial least squares discriminant analysis (PLS-DA) scatter plot of the metabolites tested in Example 1.

FIG. 6 is a 2D PLS-DA scores plot of the metabolites tested in Example 1.

FIG. 7 is a Q2, R2 and accuracy bar graph of the metabolites tested in Example 1.

FIG. 8 is a graph showing Recursive Support Vector Machines (R-SVM) data of the metabolites tested in Example 1. The data is plotted as error rate vs. number of variables (levels).

FIG. 9 shows the Receiver Operating Curve (ROC) for the SVM model with the 95% confidence interval (Example 1).

FIG. 10 shows the predicted class probabilities for the metabolites tested in Example 1.

FIG. 11 shows the top 15 metabolites of Example 1 ranked based on average importance in the Support Vector Machine (SVM) model.

FIG. 12 is a 2D PCA scores plot of the metabolites tested in Example 2.

FIG. 13 is a 2D PLS-DA scores plot of the metabolites tested in Example 2.

FIG. 14 is a Q2, R2 and accuracy bar graph of the metabolites tested in Example 2.

FIG. 15 is a frequency vs permutation test statistics graph for the metabolites tested in Example 2.

FIG. 16 shows the top 15 metabolites of Example 2 ranked based on VIP scores.

FIG. 17 shows the ROC for the SVM model with the 95% confidence interval (Example 2).

FIG. 18A shows statistical proteomic biomarker data of Example 3 for C-reactive protein. The left plot shows the original concentration and the left plot normalized concentration data.

FIG. 18B shows statistical proteomic biomarker data of Example 3 for Alpha-1-antichymotrypsin. The left plot shows the original concentration and the left plot normalized concentration data.

FIG. 18C shows statistical proteomic biomarker data of Example 3 for Serotransferrin. The left plot shows the original concentration and the left plot normalized concentration data.

FIG. 18D shows statistical proteomic biomarker data of Example 3 for Leucine-rich alpha-2-glycoprotein 1. The left plot shows the original concentration and the left plot normalized concentration data.

FIG. 18E shows statistical proteomic biomarker data of Example 3 for Serum albumin. The left plot shows the original concentration and the left plot normalized concentration data.

FIG. 18F shows statistical proteomic biomarker data of Example 3 for Coagulation factor XIII A chain. The left plot shows the original concentration and the left plot normalized concentration data.

FIG. 18G shows statistical proteomic biomarker data of Example 3 for Biotinidase. The left plot shows the original concentration and the left plot normalized concentration data.

FIG. 18H shows statistical proteomic biomarker data of Example 3 for Gelsolin. The left plot shows the original concentration and the left plot normalized concentration data.

FIG. 18I shows statistical proteomic biomarker data of Example 3 for Apolipoprotein A-IV. The left plot shows the original concentration and the left plot normalized concentration data.

FIG. 18J shows statistical proteomic biomarker data of Example 3 for Alpha-2-HS-glycoprotein. The left plot shows the original concentration and the left plot normalized concentration data.

FIG. 19 is a principal components analysis (PCA) scatter plot of the proteins tested in Example 3.

FIG. 20 is a 2D PCA scores plot of the proteins tested in Example 3.

FIG. 21 is a partial least squares discriminant analysis (PLS-DA) scatter plot of the proteins tested in Example 3.

FIG. 22 is a 2D PLS-DA scores plot of the proteins tested in Example 3.

FIG. 23 is a Q2, R2 and accuracy bar graph of the proteins tested in Example 3.

FIG. 24 is a graph showing Recursive Support Vector Machines (R-SVM) data of the proteins tested in Example 3. The data is plotted as error rate vs. number of variables (levels).

FIG. 25 shows the Receiver Operating Curve (ROC) for the SVM model with the 95% confidence interval (Example 3).

FIG. 26 shows the predicted class probabilities for the proteins tested in Example 3.

FIG. 27 shows the top 15 proteins of Example 3 ranked based on average importance in the Support Vector Machine (SVM) model.

FIG. 28 is a 2D PCA scores plot of the metabolites tested in Example 4.

FIG. 29 is a 2D PLS-DA scores plot of the metabolites tested in Example 4.

FIG. 30 is a Q2, R2 and accuracy bar graph of the metabolites tested in Example 4.

FIG. 31 is a frequency vs permutation test statistics graph for the metabolites tested in Example 4.

FIG. 32 shows the top 15 metabolites of Example 4 ranked based on VIP scores.

FIG. 33 shows the ROC for the SVM model with the 95% confidence interval (Example 4).

In the drawings, exemplary embodiments are illustrated by way of example. It is to be expressly understood that the description and drawings are only for the purpose of illustrating certain embodiments and are an aid for understanding. They are not intended to be construed as limiting to the invention in any manner.

### DETAILED DESCRIPTION

A detailed description of one or more embodiments of the invention is provided below along with accompanying figures that illustrate the principles of the invention. The invention is described in connection with such embodiments, but the invention is not limited to any particular embodiment described herein. The scope of the invention is limited only by the claims and equivalents thereof. Numerous specific details are set forth in the following description in order to provide a thorough understanding of the invention. These details are provided for the purpose of providing non-limiting examples and the invention may be practiced according to the claims without some or all of these specific details. For the purpose of clarity, certain technical material that is known in the technical fields related to the invention has not been described in detail so that the invention is not unnecessarily obscured by such descriptions.

### DEFINITIONS

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

Articles such as “a” and “an” when used in a claim, are understood to mean one or more of what is claimed or described.

The terms “Alzheimer's” and “Alzheimer's disease” are used interchangeably to generally describe Alzheimer's as identified by a qualified individual.

The term “Alzheimer's-negative” generally refers to a biological sample from an individual that does not suffer from Alzheimer's or developing Alzheimer's.

The term “Alzheimer's treatment regime” generally refers to an intervention made in response to a subject suffering from Alzheimer's. The aim of the regime may include, but is not limited to, one or more of the alleviation or prevention of symptoms, slowing or stopping the progression or worsening of Alzheimer's and the remission of Alzheimer's. In some embodiments, “Alzheimer's treatment regime” refers to therapeutic treatment (e.g., changing Alzheimer's-related metabolite levels and/or protein levels), dietary adjustments, nutritional supplements and/or cognitive training.

The terms “comprises”, “comprising”, “include”, “includes”, “including”, “contain”, “contains” and “containing” are meant to be non-limiting, i.e., other steps and other sections which do not affect the end of result can be added. The above terms encompass the terms “consisting of” and “consisting essentially of”.

The term “improvement in cognitive performance” generally refers to prevention or reduction in the severity or frequency, to whatever extent, of one or more of the behavioral disorders, symptoms and/or abnormalities expressed by individual suffering from Alzheimer's disease, or a pathological condition with one or more of the symptoms of Alzheimer's disease. Non-limiting examples of the cognitive symptoms include memory impairment and other cognitive skills, and judgement functional abilities. The improvement may be observed by the individual undertaking the treatment or by another person (i.e., medical or otherwise). Assessing whether there is an improvement may comprise conducting tests to assess memory impairment and other thinking skills, judge functional abilities, and identify behavior changes.

The term “metabolite” generally refers to any molecule involved in metabolism. Metabolites can be products, substrates or intermediates in metabolic processes. Metabolites may include, without limitation, amino acids, peptides, acylcamitines, monosaccharides, lipids and phospholipids, lysophospholipid, sphingolipids, glycerophospholipids, glucose, prostaglandins, hydroxy eicosatetraenoic acids, hydroxyoctadecadienoic acids, steroids, bile acids, and glycolipids and phospholipids.

The term “Alzheimer's-related metabolite” or “metabolomic profile” generally refers to metabolites associated with Alzheimer's disease comprising one, or two or more metabolites described herein or a combination thereof.

The terms “preferred”, “preferably” and variants generally refer to embodiments of the disclosure that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The term “preventing”, and “prevention” are used interchangeably and generally refer to any activity that leads to a reduction in risk of developing Alzheimer's disease in the subject.

The term “Alzheimer's-related protein” or “proteomic profile” generally refers to a profile of proteins, protein fragments and/or peptides associated with Alzheimer's disease comprising two or more, three or more, four or more, or five or more proteins described herein or a combination thereof. As would be appreciated by those of skill in the art, quantification of a protein can comprise quantifying a fragment or peptide thereof.

The term “subject” or “patient” is used without limitation and generally refers to a vertebrate, such as a mammal. The term “mammal” is defined as individual belonging to the class Mammalia and includes, without limitation, humans, domestic and farm animals, and zoo, sports or pet animals, such as sheep, dogs, horses, cats or cows. In some embodiments, the subject is human.

The term “treating”, or “treatment” generally refers to an intervention made in response to Alzheimer's disease or associated symptoms manifested by a subject. The aim of treatment may include, but is not limited to, one or more of the alleviation or prevention of Alzheimer's, slowing or stopping the progression or worsening of Alzheimer's and the remission of Alzheimer's. In certain embodiments, “treatment” refers to therapeutic, dietary, supplemental and/or behavior therapy.

In all embodiments of the present disclosure, all percentages, concentrations, parts and ratios are based upon the total weight of the compositions of the present disclosure, unless otherwise specified. All such weights as they pertain to listed ingredients are based on the active level and, therefore do not include solvents or by-products that may be included in commercially available materials, unless otherwise specified.

All ratios are weight ratios unless specifically stated otherwise. All temperatures are in Celsius degrees (°C), unless specifically stated otherwise. All dimensions and values disclosed herein (e.g., quantities, percentages, portions, and proportions) are not to be understood as being strictly limited to the exact numerical values recited. Instead, unless otherwise specified, each such dimension or value is intended to mean both the recited value and a functionally equivalent range surrounding that value. For example, a dimension disclosed as “40 mm” is intended to mean “about 40 mm.”

### Method of Diagnosing and Treating Alzheimer's

In one embodiment, the present disclosure relates to methods for (early) diagnosis and treatment of Alzheimer's disease, and any associated symptoms, in a subject. The disclosure is predicated, at least in part, on the identification of new metabolites and/or new proteins that provide etiological information related to Alzheimer's and provides an opportunity for objective metabolite-based and/or protein-based diagnosis of a subject's Alzheimer's that can lead to more effective therapy.

Given the complexities of the interactions between genetics and the environment, metabolic and/or proteomic profiling as described herein can provide molecular-based tests that aid in individualized treatment regimes. Metabolism and/or proteomic based analysis has the advantage to identify biomarker profiles derived from an individual's inherited genes and/or the interactions of the individual's current lifestyle behaviors (e.g., smoking, alcohol consumption, sleep behaviours, physical activity and the like), gut microbiome, dietary, and environmental factors that contribute to the unique metabolic profile and/or protein profile of a subject with Alzheimer's disease. Combining early diagnoses and an Alzheimer's treatment regime has the further advantage of increased positive therapeutic outcomes. Described herein are methods that provide for the identification of new metabolic profiles and/or proteomic profiles among subjects with Alzheimer's disease that serve to diagnose and treat those subjects. Therefore, the present disclosure provides an advancement in the art.

According to the present disclosure, a new metabolomic profile for Alzheimer's disease is identified in a subject having Alzheimer's disease. The metabolomic profile for Alzheimer's comprises at least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and Serotonin.

In another embodiment, the proteomic profile for Alzheimer's disease comprises at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV or Alpha-2-HS-glycoprotein.

In another embodiment, the proteomic profile for Alzheimer's disease comprises at least one, at least two, at least three, at least four or at least five Alzheimer's-related proteins selected from Coagulation factor XIII A Chain; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Coagulation factor X; Alpha-1-antichymotrypsin; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

In another embodiment, the proteomic profile for Alzheimer's disease comprises at least one, at least two, at least three, at least four or at least five Alzheimer's-related protein selected from Coagulation factor XIII A Chain; Complement factor D; IgGFc-binding protein; Serum amyloid A-1 protein; Ceruloplasmin; or Haptoglobin.

In another embodiment, the metabolomic profile for Alzheimer's disease comprises at least one of Coagulation factor XIII A Chain, Trigonelline, lysophosphatidylcholine (lysoPC) C18:2 fatty acid metabolite and Hydroxyphenylacetic acid (HPA).

Hydroxyphenylacetic acid (HPA) is a metabolic byproduct that inhibits the activity of important enzymes involved in neurotransmitter metabolism, leading to a decrease in neurotransmitter levels.

Trigonelline is a type of alkaloid that has anti-inflammatory and antioxidant properties. The inventors' model identified low levels of Trigonelline in Alzheimer's disease patient samples.

C18:2 and C6:1 are types of fatty acid metabolites and individuals with Alzheimer's disease tend to have higher levels of C18:2 in their blood compared to healthy individuals. Individuals with Alzheimer's disease may have altered levels of C6:1 in their blood compared to healthy individuals. C18:2 fatty acid metabolite may contribute to the formation of beta-amyloid, a protein that forms the plaques that are a hallmark of Alzheimer's disease. Additionally, C18:2 may promote inflammation in the brain, which can contribute to the death of neurons and the decline in cognitive function seen in Alzheimer's disease. The inventors' model has indicated lower levels of C18:2 and C6:1 in Alzheimer's disease samples, which is a surprising observation.

Homocysteine is an amino acid that may contribute to the development of Alzheimer's disease. Elevated levels of homocysteine may be observed in individuals with Alzheimer's disease, and high homocysteine levels have been associated with an increased risk for the development of the disease. Homocysteine has been shown to disrupt normal brain function by inhibiting the activity of enzymes involved in neurotransmitter metabolism, leading to a decrease in neurotransmitter levels and a decline in cognitive function. The inventors' model has indicated lower levels of homocysteine in Alzheimer's disease samples, which is a surprising observation.

### Proteins or Proteomic Profile

With the present disclosure, a new proteomic profile for Alzheimer's disease is identified in the subject having Alzheimer's. The proteomic profile for Alzheimer's comprises at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

According to one example, the proteomic profile for Alzheimer's disease comprises at least one, at least two, at least three, at least four or at least five Alzheimer's-related proteins selected from Coagulation factor XIII A Chain; Complement factor D; IgGFc-binding protein; Serum amyloid; A-1 protein; Ceruloplasmin; Haptoglobin; Adipocyte plasma membrane; Intercellular adhesion protein; Hemopexin; Cadherin-5; Coagulation factor X; Alpha-1-antichymotrypsin; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

According to a further example, the proteomic profile for Alzheimer's disease comprises at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; or Alpha-2-HS-glycoprotein.

According to a further example, the proteomic profile for Alzheimer's disease comprises at least one, at least two, at least three, at least four or at least five Alzheimer's-related proteins selected from Coagulation factor XIII A Chain; Complement factor D; IgGFc-binding protein; Serum amyloid A-1 protein; Ceruloplasmin; C-reactive protein; Tenascin C; Serum amyloid A-1 or Haptoglobin.

The proteomic profile for Alzheimer's disease may comprise at least two Alzheimer's-related proteins selected from Coagulation Factor XIII A Chain, Complement factor D or C-reactive protein.

Without being limited by theory, Coagulation Factor XIII chain (FXIII-A) is a protein involved in the process of blood clotting. Subjects with Alzheimer's disease may have altered levels of FXIII-A in their blood compared to healthy individuals. Additionally, FXIII-A may contribute to oxidative stress and inflammation in the brain, which can exacerbate the damage to brain cells and contribute to the progression of Alzheimer's disease.

Without being limited by theory, Serum Amyloid A1 protein (SAA1) is a protein that is primarily produced by the liver and is involved in the body's response to inflammation. The inventors' model shows that individuals with Alzheimer's disease may have altered levels of SAA1 in their blood compared to healthy individuals.

Without being limited by theory, Alpha 1 Antichymotrypsin (A1AT) is a protein that is involved in the regulation of inflammation and the clearance of proteases, which are enzymes that break down proteins. Subjects with Alzheimer's disease may have altered levels of A1AT in their blood and brain.

Adipocyte plasma membrane-associated protein (APMAP) was identified herein as an Alzheimer's associated protein and therefore may play a role in the development of Alzheimer's disease.

Surprising metabolomic profiles and/or proteomic profiles have been discovered by the inventors in a subject suffering from Alzheimer's as compared to non-Alzheimer's individual and/or an Alzheimer's-negative individual. In particular, in some embodiments, the levels of the Alzheimer's-related metabolites and/or Alzheimer's-related proteins may be altered in the circulation of the subject having Alzheimer's disease as compared to a non-Alzheimer's individual. In certain embodiments, the levels of the Alzheimer's-related metabolites and/or Alzheimer's-related proteins are altered in the blood (e.g., serum, plasma), body fluids (e.g., cerebrospinal fluid, pleural fluid, amniotic fluid, semen, or saliva), urine, and/or feces of the subject having Alzheimer's. Without wishing to be bound by theory, it is believed that the Alzheimer's-related metabolites and/or Alzheimer's-related proteins play a causative role in the development of Alzheimer's-related behaviors in the subject having Alzheimer's. Alternatively, the alteration in the level of the Alzheimer's-related metabolites and/or Alzheimer's-related proteins are caused by the Alzheimer's.

In one example, the present disclosure provides for a method for diagnosing and treating Alzheimer's disease in a subject. The method comprises step (a) providing a biological sample obtained from the subject, preferably a human. In accordance with the methods disclosed herein, any type of biological sample that originates anywhere from the body of a subject may be tested, including but not limited to, blood (including, but not limited to serum or plasma), cerebrospinal fluid (“CSF”), pleural fluid, urine, stool, sweat, tears, breath condensate, saliva vitreous humour, a tissue sample, amniotic fluid, a chorionic villus sampling, brain tissue, a biopsy of any solid tissue including tumor, adjacent normal, smooth and skeletal muscle, adipose tissue, liver, skin, hair, brain, kidney, pancreas, lung or the like may be used. Preferably, the biological sample obtained from a live subject is urine. The Alzheimer's-related metabolites and/or proteins may be extracted from their biological source using any number of extraction/clean-up procedures that are typically used in quantitative analytical chemistry.

The method further comprises step (b) measuring from the obtained sample, concentration levels of at least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin; and/or Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

In certain embodiments, the method comprises measuring at least 6, 7, 8, 9, 10, 11, 12, 13, 14, 15, 16, 17, 18, 19 or 20 Alzheimer's-related metabolites and/or proteins from the obtained sample.

In certain embodiments, the measurement of the concentration levels of the Alzheimer's-related metabolites and/or proteins may be through mass spectrometry, including but not limited to gas chromatography mass spectrometry (GC-MS) GC and liquid chromatography mass spectrometry (e.g., LC-MS, LC-MS-MS, LC-MRM, LC-SIM, and LC-SRM). Preferably, the Alzheimer's-related metabolites are measured by a spectroscopic technique, wherein the spectroscopic technique is selected from the group consisting of liquid chromatography, gas chromatography, liquid chromatography mass spectrometry, gas chromatography mass spectrometry, high performance liquid chromatography mass spectrometry, capillary electrophoresis mass spectrometry, nuclear magnetic resonance spectrometry (NMR), raman spectroscopy, and infrared spectroscopy. The measurement may also be performed under other methodology, such as for example, a colorimetric, enzymatic, immunological methodology, and gene expression analysis including, for example, real-time PCR, RT-PCT, northern analysis, and in situ hybridization.

In some embodiments, the mass spectrometry process for determining whether the Alzheimer's-related proteins are elevated comprises enzymatic or chemical digestion of the proteins or peptide fragments thereof of a sample obtained from a subject into peptide fragments. The peptide fragments are optionally separated and/or ionized and captured by mass spectrometry. The digestion may comprise a proteolytic digestion involving treating a preparation comprising the Alzheimer's-related proteins with an acid, base, or an enzyme such as trypsin or other proteolytic enzyme. One embodiment comprises a shotgun proteomics quantification in which the whole proteins in a complex mixture, such as serum, urine, and cell lysates, are hydrolyzed or otherwise cut into peptides and followed by multidimensional HPLC-MS, which aims to generate a global profile of protein mixtures as genome “shotgun” sequencing.

Thus, according to one aspect of the disclosure, there is provided a method for determining whether Alzheimer's-related proteins, or peptide fragments thereof, are elevated in a sample obtained from a subject, the Alzheimer's-related proteins or peptide fragments thereof being selected from at least one of C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor, the method comprising enzymatic or chemical digestion of the proteins or peptide fragments thereof of the sample obtained from a subject into peptide fragments to produce peptide fragments thereof; introducing a solution comprising the peptide fragments to a mass spectrometer, optionally after one or more treatments comprising liquid chromatography or other treatments, to quantify the peptide fragments; determining the concentration of peptide fragment(s) relative to a baselines, such as a standard(s) (e.g., peptide standards); and assessing whether the fragment(s) are elevated relative to the baseline or standard(s); identifying the subject as having Alzheimer's disease or a being predisposed to developing same if one or more of the peptides are elevated relative to the baseline or standard; and treating or causing the treating of the subject with an Alzheimer's disease treatment, optionally comprising an agent that is approved for use to treat Alzheimer's disease in a relevant jurisdiction.

According to one example, the peptide fragments include at least one, at least two, at least three, at least four or at least five Alzheimer's-related proteins selected from Coagulation factor XIII A Chain; Complement factor D; IgGFc-binding protein; Serum amyloid; A-1 protein; Ceruloplasmin; Haptoglobin; Adipocyte plasma membrane; Intercellular adhesion protein; Hemopexin; Cadherin-5; Coagulation factor X; Alpha-1-antichymotrypsin; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

According to a further example, the peptide fragments include at least one, at least two, at least three, at least four or at least five Alzheimer's-related proteins selected from C-reactive protein; Tenascin C; Serum amyloid A-1; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; or Alpha-2-HS-glycoprotein.

According to a further example, the peptide fragments for Alzheimer's disease comprise at least one, at least two, at least three, at least four or at least five Alzheimer's-related proteins selected from Coagulation factor XIII A Chain; Complement factor D; IgGFc-binding protein; Serum amyloid A-1 protein; Ceruloplasmin; C-reactive protein; Tenascin C; Serum amyloid A-1 or Haptoglobin.

In a further embodiment, the peptide fragments include one or two Alzheimer's-related proteins selected from Coagulation factor XIII A Chain, Complement factor D or C-reactive protein.

In certain embodiments, with any of the methods described herein, the methods may further include measuring the concentration levels of one or more additional Alzheimer's-related metabolites and/or proteins, including, but not limited to, any of those described herein may also be measured. The novel approach of the present disclosure identifies biomarkers that have high predictive value for a subset of the diagnostic class (i.e., Alzheimer's in this case). In one embodiment, the advantage of including additional Alzheimer's-related metabolites and/or proteins is to give rise to the opportunity to reveal additional metabolite sub-types and/or increase the overall sensitivity of a known diagnostic method. Non-limiting examples of additional Alzheimer's-relates metabolites and/or proteins are beta-amyloid 42, tau and phospho-tau. The subject is identified as having Alzheimer's if the concentration level of the one or more additional Alzheimer's-related metabolites and/or proteins and other biomarkers obtained from the biological sample is different to that in a reference Alzheimer's-negative sample. Non-limiting examples of additional Alzheimer's disease biomarkers are described in U.S. Pat. Nos. 10,914,749 and 9,285,374, and U.S. 2021/10109115, the relevant contents of which are incorporated herein by reference.

The method described herein further comprises step (c) comparing the concentration levels of the Alzheimer's-related metabolites and/or proteins from the obtained sample to the concentration levels of reference Alzheimer's-related metabolites and/or proteins from an Alzheimer's-negative sample. One skilled in the art will appreciate that references can be established as a value representative of the level of Alzheimer's-related metabolites and/or proteins in a non-Alzheimer's population that do not suffer from Alzheimer's for the comparison. Various criteria may be used to determine the inclusion and/or exclusion of a particular subject in the reference population, including age of the subject (e.g., the reference subject can be within the same age group as the subject in need of treatment) and gender of the subject (e.g., the reference subject can be the same gender as the subject in need of treatment). In certain embodiments, the reference is from an Alzheimer's-negative sample obtained from a non-Alzheimer's adult aged about 65 years or more.

The method described herein further comprises step (d) identifying the subject as having Alzheimer's disease if the concentration levels of the Alzheimer's-related metabolites and/or proteins from the obtained sample are different relative to the concentration levels of the reference.

In certain embodiments, the identifying step (d) occurs upon determination that the concentration level of the at least one Alzheimer's-related metabolite and/or protein from the obtained sample differs by about 20% or more, about 30% or more, about 40% or more, about 50% or more, about 60% or more, or about 70% or more relative to the concentration level of the at least one reference Alzheimer's-related metabolites and/or proteins from the Alzheimer's-negative sample. In certain embodiments, the identifying step (d) occurs upon determination that the concentration levels of at least two, at least three, at least four or at least five Alzheimer's-related metabolites and/or proteins from the obtained sample differ by about 20% or more, about 30% or more, about 40% or more, about 50% or more, about 60% or more, or about 70% or more relative to the concentration levels of the reference Alzheimer's-related metabolites and/or proteins from the Alzheimer's-negative sample.

In certain embodiments, the identifying step (d) occurs upon determination that the concentration levels of the homocysteine, C 18:2 and/or C6:1 are decreased relative to the concentration levels of the reference from the Alzheimer's-negative sample.

In some aspects, the concentration level of the homocysteine, C 18:2 and/or C6:1 from the obtained sample is lower than about 20% or more, about 30% or more, about 40% or more, about 50% or more, about 60% or more, or about 70% or more relative to the concentration level of the reference homocysteine, C 18:2 and/or C6:1 from the Alzheimer's-negative sample.

In certain embodiments, the identifying step (d) occurs upon determination that the concentration level of one or more of Hydroxyphenylacetic acid; Trigonelline; Coagulation factor XIII A Chain; or Complement factor D from the obtained sample is increased relative to the concentration level of the Hydroxyphenylacetic acid; Trigonelline; Coagulation factor XIII A Chain; or Complement factor D from the Alzheimer's-negative sample. In some aspects, the concentration level of the Hydroxyphenylacetic acid; Trigonelline; Coagulation factor XIII A Chain; or Complement factor D from the obtained sample is elevated by about 20% or more, about 30% or more, about 40% or more, about 50% or more, about 60% or more, or about 70% or more relative to the concentration level of the Hydroxyphenylacetic acid; Trigonelline; Coagulation factor XIII A Chain; or Complement factor D from the Alzheimer's-negative sample.

The method described herein further comprises step (e) treating the subject so identified as having Alzheimer's with an Alzheimer's treatment regime.

In certain embodiments, with any of the methods described herein, the comparison of the concentration level of the at least one Alzheimer's-related metabolite and/or protein from the obtained sample to the concentration level of the reference Alzheimer's-related metabolite and/or protein from the Alzheimer's-negative sample comprises using multivariate statistical analysis. Preferably, the multivariate statistical analysis is selected from principal component analysis (“PCA”), or partial least squares projects to latent structures discriminant analysis (“PLS-DA”). In certain embodiments, a computer is used for statistical analysis. Data for statistical analysis can be extracted from chromatograms (i.e., spectra of mass signals) using software for statistical methods known in the art.

In some aspects, the present disclosure relates to a method of monitoring Alzheimer's disease progression and treating Alzheimer's in a subject. In some embodiments, the method includes quantifying the Alzheimer's-related metabolites and/or proteins at one or more time points after the initiation of treatment to monitor Alzheimer's progression (e.g., rate of decline or rate of improvement of Alzheimer's progression) in a subject.

Accordingly, the method comprises: (a) providing a first biological sample obtained from the subject at a first time; (b) assessing a first Alzheimer's-related metabolite profile by measuring concentration levels of at least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor; from the first obtained sample; (c) comparing the first Alzheimer's-related metabolite and/or proteomic profile with a reference Alzheimer's-related metabolite profile from an Alzheimer's-negative sample; (d) determining that there is a first difference between the first Alzheimer's-related metabolite and/or proteomic profile and the reference Alzheimer's-related metabolite and/or proteomic profile from the Alzheimer's-negative sample, the first difference being indicative of Alzheimer's; (e) providing a second biological sample obtained from the subject at a second time that is after the first time; (f) assessing a second Alzheimer's-related metabolite profile and/or proteomic profile by measuring concentration levels of the Alzheimer's-related metabolites and/or proteins from the second obtained sample; (g) comparing the second Alzheimer's-related metabolite and/or proteomic profile with the reference Alzheimer's-related metabolite and/or proteomic profile from the Alzheimer's-negative sample; (h) determining that there is a second difference between the first Alzheimer's-related metabolite profile and/or proteomic profile and the reference Alzheimer's-related metabolite and/or proteomic profile from the Alzheimer's-negative sample, the second difference being indicative of Alzheimer's; (i) determining Alzheimer's progression based on at least in part on the first and second difference; and (j) treating the subject as identified with an Alzheimer's treatment regime.

In certain embodiments of the above method, the period between the first time and the second time is at least 1 month, at least 2 months, at least 3 months, at least 6 months, at least 9 months, or at least 12 months, preferably at least 3 months. In some embodiments, the treatment has been administered to the subject before the first two biological samples have been obtained. In other embodiments, the treatment has been administered to the subject in the interval(s) between the taking of the biological samples. In certain embodiments, the first biological sample, the second biological sample, or both are blood or urine, preferably serum, plasma or urine.

According to embodiments of the above method, the metabolomic profile for Alzheimer's disease comprises at least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin.

In another embodiment, the metabolomic profile for Alzheimer's disease comprises at least one, at least two, at least three, at least four or at least five Alzheimer's-related metabolites selected from C-reactive protein; Tenascin C; Serum amyloid A-1; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV or Alpha-2-HS-glycoprotein.

In another embodiment, the metabolomic profile for Alzheimer's disease comprises at least one, at least two, at least three, at least four or at least five Alzheimer's-related metabolites selected from Coagulation factor XIII A Chain; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Coagulation factor X; Alpha-1-antichymotrypsin; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

In another embodiment, the metabolomic profile for Alzheimer's disease comprises at least one, at least two, at least three, at least four or at least five Alzheimer's-related metabolites selected from Coagulation factor XIII A Chain; Complement factor D; IgGFc-binding protein; Serum amyloid; A-1 protein; Ceruloplasmin; or Haptoglobin.

In another embodiment, the metabolomic profile for Alzheimer's disease comprises at least one of Coagulation factor XIII A Chain and Hydroxyphenylacetic acid.

According to embodiments of the above method, the proteomic profile for Alzheimer's disease comprises at least one, at least two, at least three, at least four or at least five Alzheimer's-related proteins selected from Coagulation factor XIII A Chain; Complement factor D; IgGFc-binding protein; Serum amyloid; A-1 protein; Ceruloplasmin; Haptoglobin; Adipocyte plasma membrane; Intercellular adhesion protein; Hemopexin; Cadherin-5; Coagulation factor X; Alpha-1-antichymotrypsin; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor.

According to embodiments of the above method, the proteomic profile for Alzheimer's disease comprises at least one, at least two, at least three, at least four or at least five Alzheimer's-related proteins selected from C-reactive protein; Tenascin C; Serum amyloid A-1; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; or Alpha-2-HS-glycoprotein.

According to embodiments of the above method, the proteomic profile for Alzheimer's disease comprises at least one, at least two, at least three, at least four or at least five Alzheimer's-related proteins selected from Coagulation factor XIII A Chain; Complement factor D; IgGFc-binding protein; Serum amyloid A-1 protein; Ceruloplasmin; C-reactive protein; Tenascin C; Serum amyloid A-1 or Haptoglobin.

According to the above method, the proteomic profile for Alzheimer's disease may comprise at least two Alzheimer's-related proteins selected from Coagulation factor XIII A Chain, Complement factor D or C-reactive protein.

In certain embodiments of the above-described method, the concentration levels of the homocysteine, C 18:2 and/or C6:1 are decreased relative to the concentration levels of the reference from the Alzheimer's-negative sample.

In some examples, the concentration level of the homocysteine, C 18:2 and/or C6:1 from the obtained sample is lower than about 20% or more, about 30% or more, about 40% or more, about 50% or more, about 60% or more, or about 70% or more relative to the concentration level of the reference homocysteine, C 18:2 and/or C6:1 from the Alzheimer's-negative sample.

### Alzheimer's Treatment Regime

The Alzheimer's treatment regime is selected from the group consisting of dietary adjustment, nutritional supplement or a combination thereof, to the subject diagnosed as having or predisposed for developing Alzheimer's. Preferably, the Alzheimer's treatment regime has the effect of adjusting the concentration levels of one or more of the Alzheimer's-related metabolites and/or proteins in the subject diagnosed as having or predisposed of developing the Alzheimer's towards the corresponding levels of the reference Alzheimer's-related metabolites and/or proteins from the Alzheimer's-negative sample.

Various methods can be used to adjust the concentration level, for example blood level (e.g., serum level), of the Alzheimer's-related metabolite and/or protein in the subject. Preferably, the adjustment of the concentration level of the one or more Alzheimer's-related metabolites in the subject occurs until an improvement in the behavioral performance in the subject is observed.

In certain embodiments, an antibody that specifically binds the Alzheimer's-related metabolite, an intermediate for the in vivo synthesis of the Alzheimer's-related metabolite, or a substrate for the in vivo synthesis of the Alzheimer's-related metabolite can be administered to the subject. For example, an antibody that specifically binds one or more of metabolites and/or proteins on the metabolomic and/or proteomic profile can be used to reduce the levels thereof in the subject.

In certain embodiments, the concentration level, for example blood level (e g., serum level), of the one or more Alzheimer's-related metabolites is adjusted by adjusting the composition of gut microbiota in the subject.

In further embodiments, nucleic acid therapy can be used to reduce the concentration of a protein (which includes a peptide) by using, for example, siRNA or antisense oligonucleotides to reduce expression of the protein. Likewise, nucleic acid therapy can be used to express a protein that is present at lower levels in the Alzheimer's disease-free reference using mRNA therapy. The therapeutic nucleic acid can be encapsulated in a suitable delivery vehicle. Nucleic acid therapy can also be used to increase the level of a metabolite described herein by using, for example, mRNA, antisense or siRNA therapy by, for example, modulating the activity of a protein that is involved in metabolism.

In another embodiment, the subject diagnosed or identified as being predisposed to developing Alzheimer's disease is treated or caused to be treated with an approved Alzheimer's therapeutic, such as a drug. The drug may be approved by any applicable regulator. Non-limiting examples of Alzheimer's drugs include Galantamine, rivastigmine, and donepezil are cholinesterase inhibitors that are prescribed for mild to moderate Alzheimer's symptoms. These drugs may help reduce or control some cognitive and behavioral symptoms.

### Kits

The metabolomic and/or proteomic profile described herein may be utilized in tests, assays, methods, kits for diagnosing, predicting, modulating or monitoring Alzheimer's disease, including ongoing assessment, monitoring and/or susceptibility assessment. The present disclosure includes a kit for diagnosis of Alzheimer's by measuring and identifying at least one or more Alzheimer's-related metabolites and/or protein associated with Alzheimer's. Preferably, the kit may comprise appropriate Alzheimer's treatment regime to be initiated upon the determination of Alzheimer's. Accordingly, the kit comprises (a) a detector configured to detect concentration levels of at least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin; and/or at least one, at least two, at least three, at least four or at least five Alzheimer's-related proteins (e.g., peptides) selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor control levels corresponding to control group of Alzheimer's-negative subjects, (c) a multivariate analysis system configured to analyze a difference in the concentration levels of the Alzheimer's-related metabolites and/or proteins and the control levels, and (d) optionally, instruction for an Alzheimer's diagnosis method, wherein the method comprises measuring, using the detector, the levels of the Alzheimer's-related metabolites and/or proteins from the obtained biological sample, and comparing the levels of the obtained Alzheimer's-related metabolites and/or proteins to the control levels of the Alzheimer's-related metabolites and/or proteins obtained from Alzheimer's-negative subjects. Preferably, the Alzheimer's diagnosis method comprises a multi-metabolite or protein (e.g., peptide) detector configured to measure the levels of Alzheimer's-related metabolites and/or proteins.

In some aspects, the kit may be for the measurement of the Alzheimer's-related metabolites and/or proteins by a physical separation technique (as described herein above). In some aspects, the kit may be for measurement of the Alzheimer's-related metabolites and/or proteins by a methodology other than a physical separation method, such as for non-limiting example, a colorimetric, enzymatic, and immunological methodology. The kit may also include one or more appropriate negative and/or positive controls. Kit of the present disclosure may include other reagents such as buffers and solutions needed to perform the tests.

### Computer-Implemented Method

In a further embodiment, the metabolite and/or proteome profile measured is based on a profile identified in a previous computer-implemented statistical analysis model that has a predictive value of at least 80%, 85%, 90%, 92%, 94% or 96% and comprises at least one of means comparison, PCA, PLS-DA or recursive SVM data analyses.

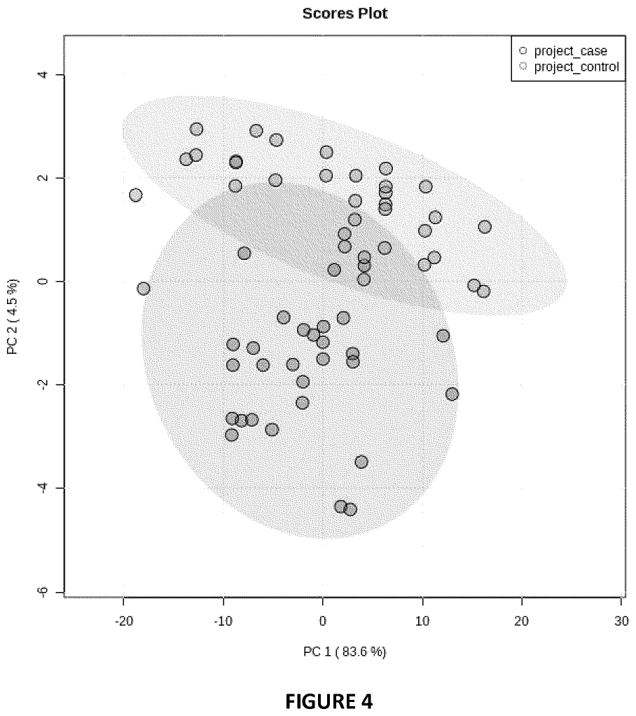

In a further embodiment, the metabolite and/or proteome profile is identified as having the predictive value by classifying the samples into the two groups by a computer-implemented method based on the predicted metabolites and/or proteins used in the computer model, wherein the data from two groups are sufficiently separated, such as on a scores plot (e.g., see Examples below and FIGS. 4, 6 and 16 that exemplify separation of a control and model data).

The disclosure is also directed to a computer-implemented method for processing a biological sample of a subject, diagnosing an Alzheimer's and treating (or causing treatment thereof) of the subject diagnosed with Alzheimer's disease. The computer-implemented method may further allow monitoring of Alzheimer's progression across multiple time points to support a more effective treatment regime.

The computer-implemented method comprises receiving a biological sample from the subject; processing the sample in a spectroscopy unit directly or wirelessly linked, or may utilize any suitable communication technology, to a processing device, the processing device having memory for storing measurement data from the spectroscopy unit; and in the spectroscopy unit, measuring levels of least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor; and storing the measurement data in the processor. The processing device comprises one or more data storage devices that may be configured or adapted to store data related to the method. For example, the data storage device may be configured or adapted to store measurement data from the spectroscopy unit. The data storage device may also comprise computer program code stored thereon. The program code of this embodiment may include program code for at least performing the steps of the method aspect upon execution thereof.

The computer-implemented method further comprises comparing the stored measurement data to a value in the memory representing an Alzheimer's-negative sample using multivariate statistical analysis; storing on the processing device a result corresponding to at least one, at least two, at least three, at least four or at least five Alzheimer's-related metabolites selected from Hydroxyphenylacetic acid; Trigonelline; a Lysophosphatidylcholine (lysoPC) selected from a C18:2, C18:0, C18:1, C16:0, C14:0, C16:1, C20:4, C17:0 or C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline, Cotinine, Glucose, C3-DC)+3-Hydroxybutyrylcarnitine (C4-OH) (C3-DC (C4-OH)) and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor from the obtained sample, wherein the result identifies the subject as having Alzheimer's if the measurement data representing the level of the Alzheimer's-related metabolite and/or protein is different relative to a concentration value of a reference Alzheimer's-related metabolite and/or protein from an Alzheimer's-negative sample; and displaying an Alzheimer's treatment regime on an electronic display connected directly or wirelessly to the processor for the subject identified as having Alzheimer's or as having predisposition of developing Alzheimer's. The displayed treatment regime comprises electronic text, optionally with graphical icons, on a graphical user interface describing one or more of: dietary adjustments, nutritional supplements, behavior training or a combination thereof, to the subject diagnosed as having or predisposed of developing the Alzheimer's, or adjusting the blood levels of one or more of the Alzheimer's-related metabolites and/or proteins in the subject diagnosed as having or predisposed of developing the Alzheimer's until an improvement in the cognitive and/or behavioral performance in the subject is observed; preferably the adjustment of the blood levels of one or more of the Alzheimer's-related metabolites comprises adjusting the composition of gut microbiota in the subject.

In one embodiment the graphical user interface comprises a dashboard with the graphical icons describing one or more treatment regimes.

### EXAMPLES

The following examples describe some exemplary modes of practicing certain methods that are described herein. It should be understood that the examples are for illustrative purposes only and are not meant to limit the scope of the systems and methods described herein.

Supervised machine learning is a type of artificial intelligence that can be used to build predictive models for disease prediction. The process uses a large dataset of labeled patient information, which can include demographic information, medical and family history, and clinical results, to train a machine learning algorithm to identify patterns and relationships that are associated with a particular risk of developing that disease. Once the model has been trained, it can be used to make predictions about new patients based on their demographic and clinical information.

The inventors utilized this process to generate a machine learning model for Alzheimer's disease prediction based on Alzheimer's disease and health control patient samples from biobanks and used both demographic, clinical, and molecular (metabolomics, proteomics) data to predict the likelihood of a patient developing Alzheimer's disease. The inventors' model identifies a signature of metabolites and proteins which can be used to classify new patients into Alzheimer's disease risk class or healthy control class with a 96% degree of accuracy. The models have also identified several proteins which are associated with disease mechanisms and provide valuable insights as candidate targets for therapeutic interventions to prevent or delay the onset of disease.

The identification of the novel metabolomic/proteomic panels was conducted in two separate studies comparing the proteins and metabolites of Alzheimer's patients and control groups. The novel metabolomic profiles in study 1 and study 2 respectively are described in Example 1 and 2. The novel proteomic profiles in study 1 and study 2 are set forth in Example 3 and 4, respectively.

### Below Level of Detection (BLQ) and Above the Limit of Detection (ALQ) Values

In order to maximize the number of samples and biomarkers included in the analysis (i.e., reduce missingness in the data), concentration values that were below the limit of detection (BLQ) and above the limit of quantification (ALQ) were processed prior to analysis.

Measures marked as BLQ were assigned a random concentration value between 0 and the lower limit of detection/quantification (LLOD/LLOQ). Measures marked as ALQ were assigned a concentration value that was 1.5*the upper limit of quantification (ULOQ).

For proteomics, in some instances the ALQ entries differ in the raw data for the same protein. To account for these discrepancies, the ‘universal’ upper limit of quantification (ULOQ) value from the batch was used to calculate the concentration value for biomarkers marked as ALQ.

### Missing Values

After processing the BLQ and ALQ values, the metabolomics data did not contain any missing values and therefore it was not necessary to drop further samples or biomarkers from analysis.

### Scaling and Transformations

No sample normalization was performed. Data was log (base 10) transformed to account for the right-skew nature of biological data. Data was scaled to be mean-centered and divided by the standard deviation of each biomarker for classification modeling.

### Demographics

In total, 63 samples were included in the analysis for Examples 1 and 4.

### Age Distribution and Sex

### Example 1: Metabolomics Study 1

In a first metabolomic study, there were 150 metabolites included in the analysis, of which 72 biomarkers showed a significant difference between the case and control groups using the two-sample t-test for means comparison. The false-discovery rate was accounted and adjusted for.

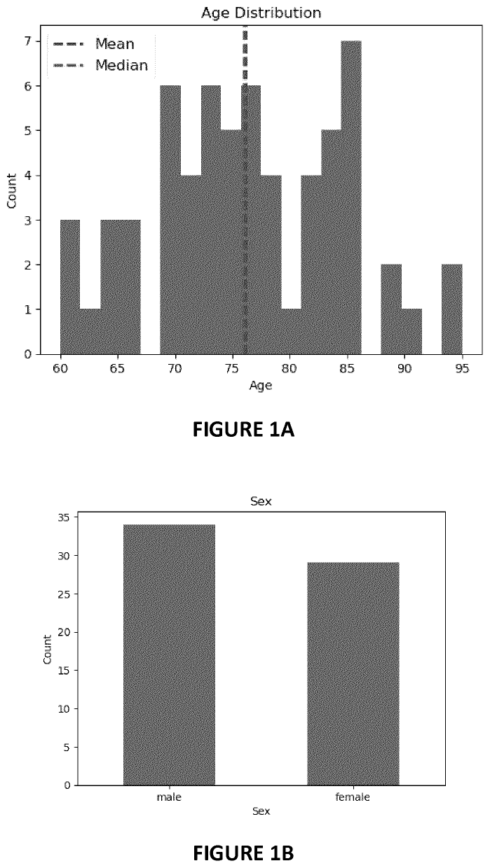

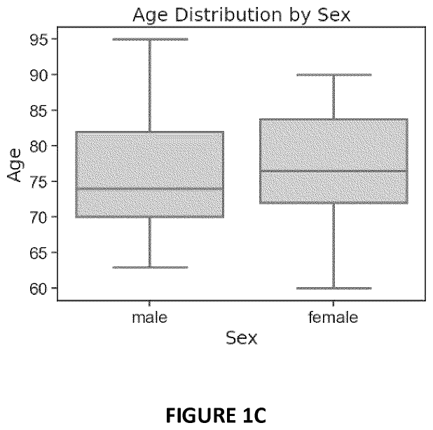

The age in the cohort ranges from 60 to 95 and generally was evenly distributed with a mean age of 76.1 and a median age of 76.0. The youngest participant was 60 years old, and the oldest was 95 (FIG. 1A). There were a similar number of males (n=34) and females (n=29) in the cohort (FIG. 1B). Though females in the cohort tended to have a younger range of age, the average median age is slightly higher compared to males (FIG. 1C).

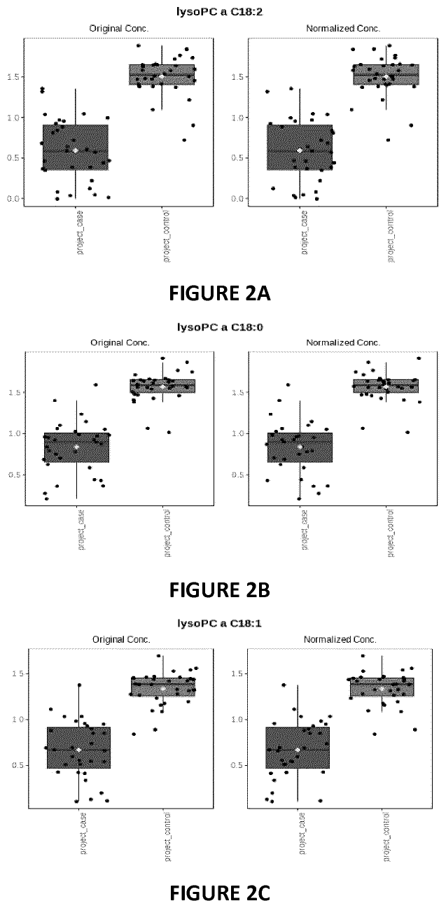

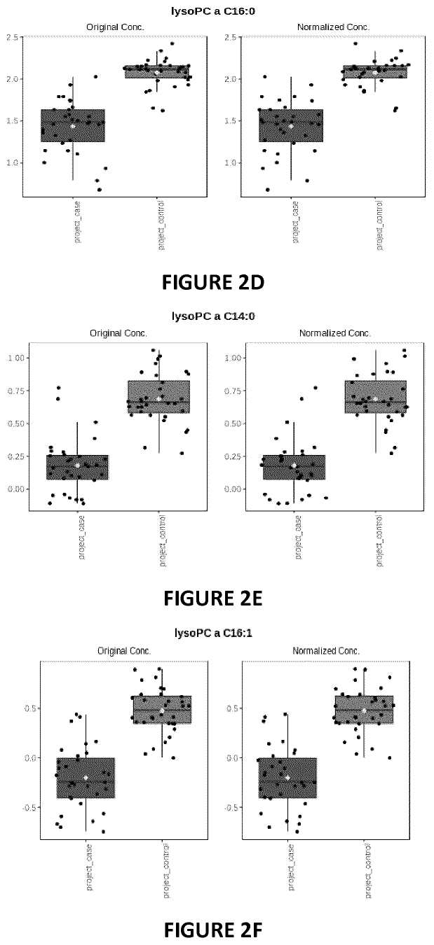

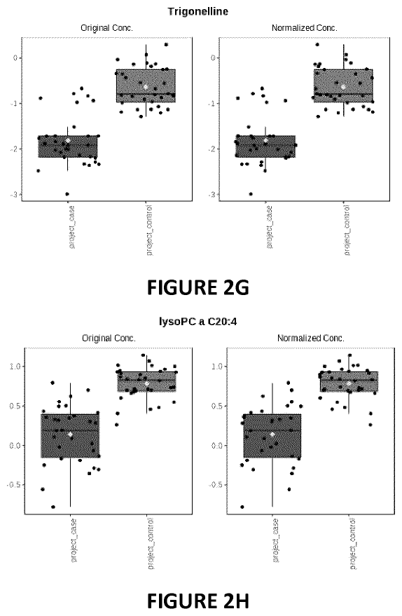

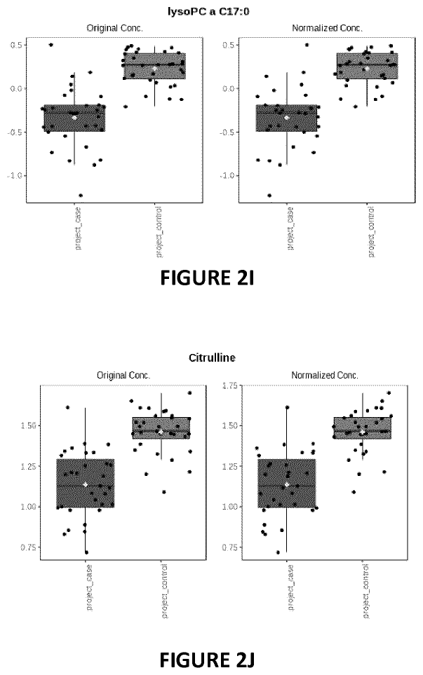

The 10 metabolites with the lowest p-values are provided in Table 1 below and the results of the statistical analysis are depicted graphically in FIG. 2A-J.

TABLE 1
Metabolites with the lowest p-values in study 1

| Biomarker | P-Value | FIG. |
|---|---|---|
| Lysophosphatidylcholine | 2.2519E−16 | 2A |
| (lysoPC) a C18:2 | | |
| lysoPC a C18:0 | 2.3667E−16 | 2B |
| lysoPC a C18:1 | 5.9498E−15 | 2C |
| lysoPC a C16:0 | 1.1552E−14 | 2D |
| lysoPC a C14:0 | 1.8985E−14 | 2E |
| lysoPC a C16:1 | 7.5686E−14 | 2F |
| Trigonelline | 2.1828E−13 | 2G |
| lysoPC a C20:4 | 7.9732E−12 | 2H |
| lysoPC a C17:0 | 4.5605E−11 | 2I |
| Citrulline | 3.4491E−10 | 2J |

The study identified a variety of different lysophosphatidylcholines (LPCs) as predominant metabolites associated with Alzheimer's disease. LPCs are a type of phospholipid that are involved in the development of Alzheimer's disease. The precise mechanisms by which LPCs contribute to the development of Alzheimer's disease are still being studied, but the inventor's finding suggest that LPCs play an important role in the disease process and may be a promising target for the development of new treatments.

### Principal Component Analysis (PCA)

A PCA scores plot is a graphical representation of the results of a principal component analysis (PCA) on a dataset. PCA is a statistical technique that is used to reduce the dimensionality of a dataset by identifying and removing redundant or correlated variables and projecting the data onto a smaller number of orthogonal (uncorrelated) dimensions, called principal components.

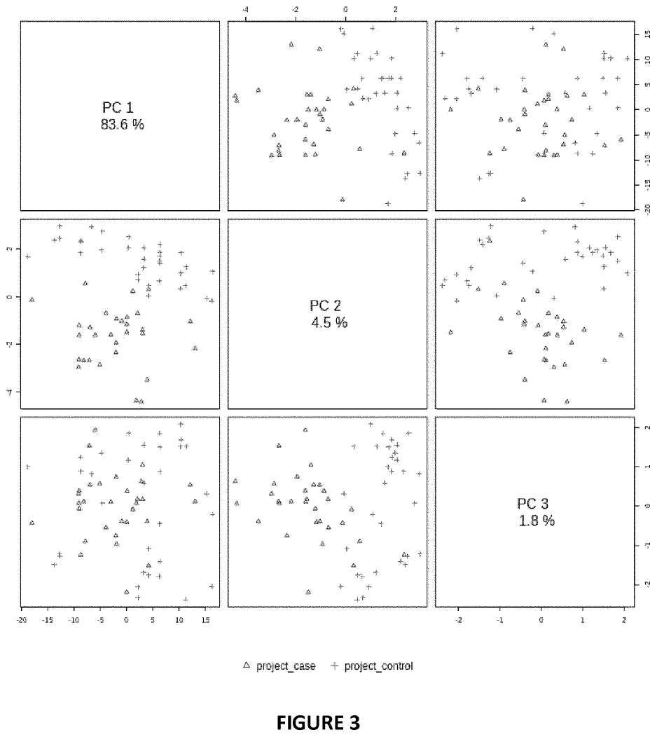

A PCA scores plot is a scatterplot that shows the projection of the data onto the first two principal components. Each point on the plot represents a single data point from the original dataset, and the position of the point reflects the values of the data point on the two principal components. The x-axis of the plot represents the first principal component, and the y-axis represents the second principal component. The PCA scatter plot is shown in FIG. 3.

The PCA scores plot can be used to visualize the structure and relationships within the data. For example, if the data points cluster together in distinct groups on the plot, it may indicate that there are underlying subgroups or clusters in the data. The PCA scores plot can also be used to identify outliers or unusual data points, and to assess the amount of variability explained by the first two principal components.

Based on the PCA 2D scores plot, there appears to be a separation between biomarkers in the AD-case group and the control group, suggesting that a new biosignature can be identified for this disease (FIG. 4).

### Partial Least Squares Discriminant Analysis (PLS-DA)

Principal component analysis (PCA) and partial least squares discriminant analysis (PLS-DA) are both dimensionality reduction techniques that are used in data analysis and machine learning. However, there are some key differences between the two methods.

PCA is an unsupervised dimensionality reduction technique that identifies and removes redundant or correlated variables in a dataset, and projects the data onto a smaller number of orthogonal dimensions. PLS-DA is a supervised learning technique that combines partial least squares regression (PLS) and linear discriminant analysis (LDA) to classify data into different classes.

The main differences between PCA and PLS-DA are that PCA is a linear technique based on the variance of the data, while PLS-DA is a non-linear technique based on the relationship between predictor and response variables. PCA is used to find principal components that explain the variation in the data, while PLS-DA is used to find latent variables that best discriminate between classes.

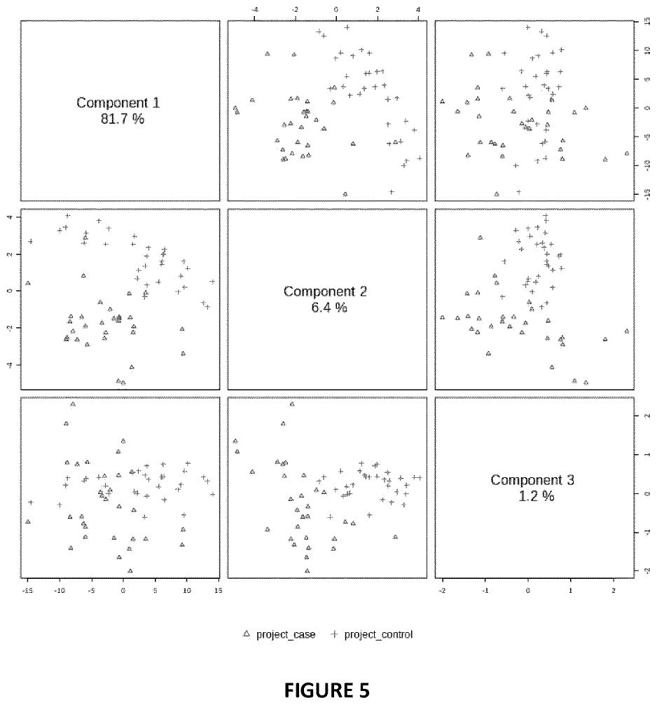

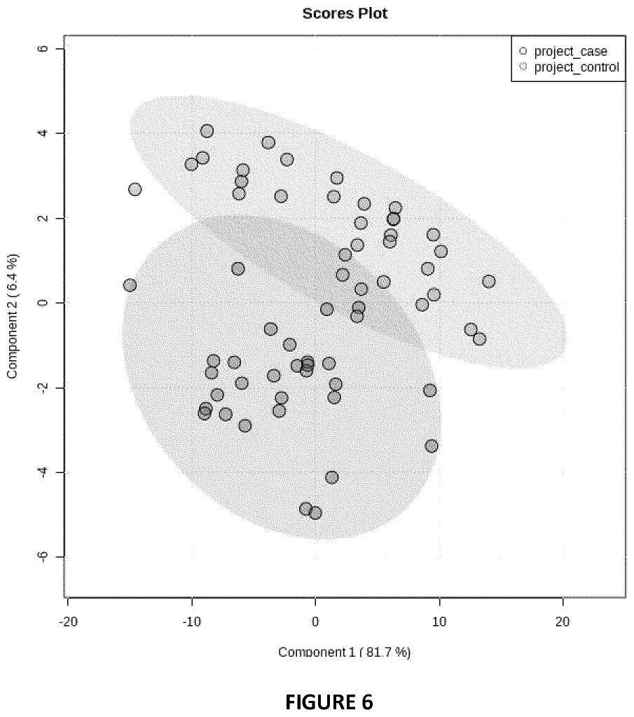

Similar to the PCA analysis, there does appear to be some separation in metabolites using PLS-DA. In general, this suggests that the PLS-DA model has a good ability to classify the samples into the two groups based on the predictor metabolites used. (FIGS. 5 and 6).

### Q2 and R2 Analysis

Q2, R2 and accuracy are three metrics that are used to evaluate the performance of a model or prediction. These metrics can be used to assess the ability of a PLS-DA model to classify data into different classes or categories.

Q2 is a measure of the predictive ability of the model. It is calculated by dividing the sum of the squares of the differences between the observed and predicted values of the response variable by the total sum of squares. A high Q2 value indicates that the PLS-DA model has a strong ability to predict the class membership of new data points.

R2 is a measure of the goodness of fit of the PLS-DA model. It is calculated by dividing the sum of the squares of the differences between the observed and predicted values of the response variable by the total sum of squares. A high R2 value indicates that the PLS-DA model explains a large portion of the variation in the response variable.

Accuracy is a measure of the degree to which the PLS-DA model correctly predicts the class membership of data points. It is calculated by dividing the number of correct predictions made by the model by the total number of predictions made. A high accuracy indicates that the PLS-DA model makes a large number of correct predictions.

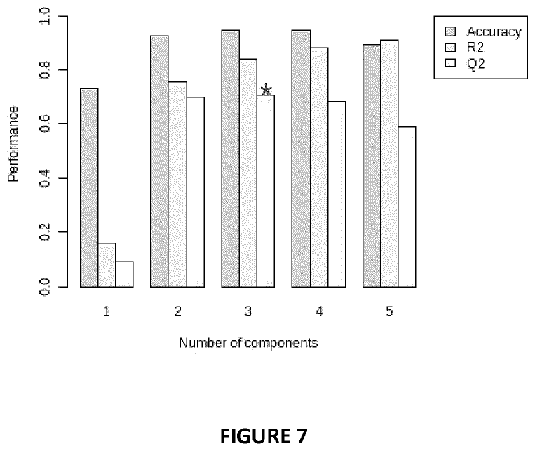

In general, a PLS-DA model with high values of Q2, R2, and accuracy is considered to be a good model, as it has a strong ability to predict the class membership of new data points and explains a large portion of the variation in the response variable. However, the appropriate values of these metrics will vary depending on the specific context and the goals of the analysis. The results are shown in FIG. 7.

### Recursive SVM (R-SVM)

R-SVM (Recursive SVM) is a feature selection algorithm that uses Support Vector Machines (SVMs) to classify data. It starts by selecting all the features in the data and using them to train an SVM model. Then, it recursively eliminates the least important features, as determined by their relative contribution to the classification using cross-validation error rates. This process creates a series of SVM models, each one using a different subset of features. The features used by the best model are then identified as the most informative ones. This approach is particularly useful when the number of features is large, and some of them are irrelevant or redundant. By eliminating the irrelevant features, R-SVM can improve the accuracy and computational efficiency of the model.

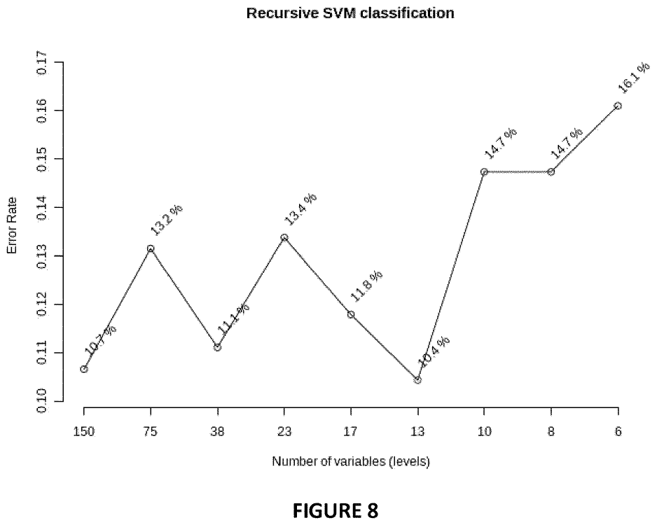

The results of the Recursive SVM classification for the metabolites is shown in FIG. 8.

### Linear Support Vector Machines (SVM)

Linear SVM is used as the classification model. Support Vector Machines (SVMs) are a type of supervised machine learning algorithm that can be used for classification. Linear SVMs, in particular, are a variant of SVMs that use a linear boundary to separate the different classes in the data.

Linear SVM finds the best boundary, called a hyperplane, that separates the different classes in the data. Once the best hyperplane is found, it can be used to classify new data points by determining which side of the boundary they fall on.

Linear SVM has some advantages over other classification algorithms. It's efficient, effective in high dimensional spaces, memory-efficient, and less prone to overfitting.

This model using metabolomics data has an average predictive accuracy of 89.6%.

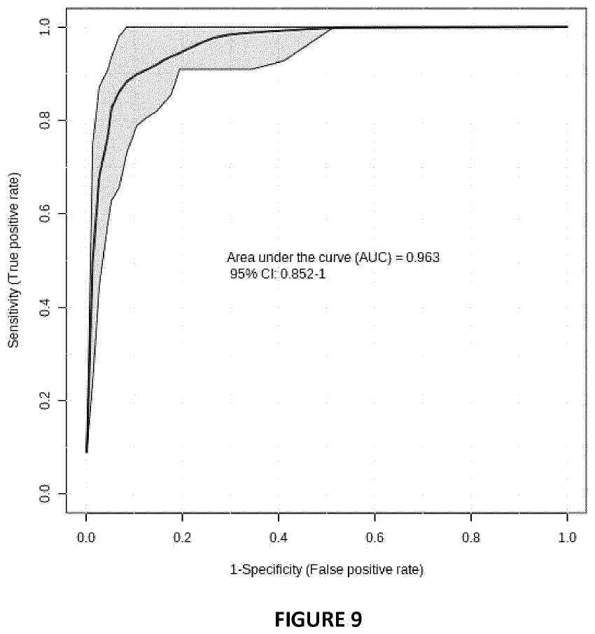

FIG. 9 shows the Receiver Operating Curve (ROC) for the SVM model with the 95% confidence interval. The area under the curve (AUC) is 96.3%. ROC is a graphical representation of the performance of a binary classification model. It is plotted on a two-dimensional graph with the true positive rate on the y-axis and the false positive rate on the x-axis. A model with a higher true positive rate and a lower false positive rate will have a better performance.

AUC and predictive accuracy are two different measures of the performance of a binary classification model.

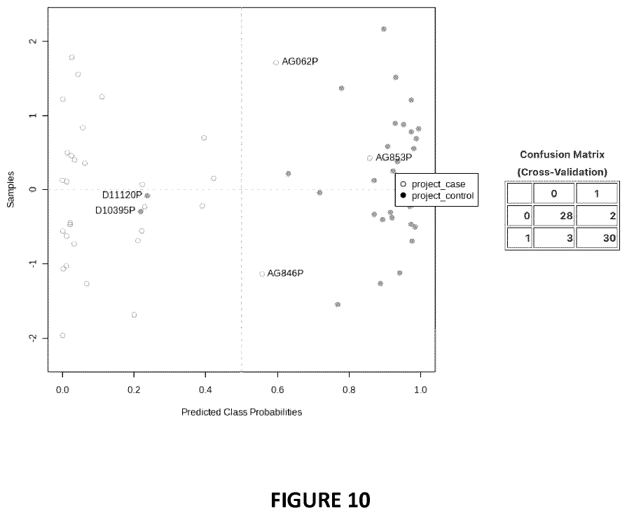

Predictive accuracy (average of 89.6% in this case) is a measure of how well a model can correctly classify new data points into the correct class. It is calculated as the proportion of correct predictions made by the model. Predictive accuracy is a simple and intuitive measure of performance, but it can be sensitive to changes in the class distribution of the data. FIG. 10 shows the predicted class probabilities.

AUC (96.3% in this case), on the other hand, is a measure of the separability of the classes in the data. It is calculated as the area under the Receiver Operating Characteristic (ROC) curve, which plots the true positive rate (sensitivity) against the false positive rate (1-specificity) at various threshold settings. AUC is a single value between 0 and 1 that represents the overall performance of the model, regardless of the decision threshold. AUC is insensitive to changes in the class distribution and it does not rely on a specific decision threshold.

While predictive accuracy is a measure of how many predictions were made correctly, AUC is a measure of how well the model is able to separate the two classes.

To summarize, in the context of separating Alzheimer's disease (positive class) from healthy (negative class) individuals, the model is able to correctly classify (on average) new data points to the correct class 89.6% of the time. In addition, an AUC of 96.3% would indicate that the model is able to correctly classify individuals with Alzheimer's disease as positive and healthy individuals as negative in 96.3% of the cases. This means that the model is able to correctly separate the two classes in most cases, and thus it would be considered to have good performance.

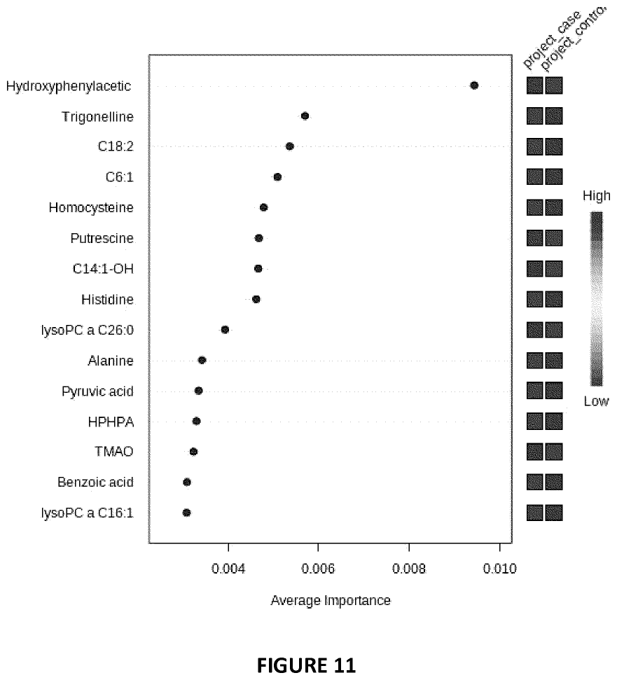

The top 15 metabolites ranked based on average importance in the SVM model are shown in FIG. 11.

The metabolites are summarized in Table 2 below.

TABLE 2
Top 15 metabolites identified by machine learning in study 1

| | | Higher/Lower than |
|---|---|---|
| No. | Alzheimer's Metabolite | control samples |
| 1 | Hydroxyphenylacetic acid (HPA) | Higher |
| 2 | Trigonelline | Lower |
| 3 | C18:2 | Lower |
| 4 | C6:1 | Lower |
| 5 | Homocysteine | Lower |
| 6 | Putrescine | Higher |
| 7 | C14:1-OH | Higher |
| 8 | Histidine | Lower |
| 9 | LysoPC a C26:0 | Higher |
| 10 | Alanine | Lower |
| 11 | Pyruvic acid | Lower |
| 12 | 3-(3-hydroxyphenyl)-3- | Lower |
| | hydroxypropionic acid (HPHPA) | |
| 13 | Trimethylamine N-oxide (TMAO) | Higher |
| 14 | Benzoic acid | Lower |
| 15 | LysoPC a C16:1 | Lower |

The top 5 metabolites are discussed below.

The inventors' model identified Hydroxyphenylacetic acid (HPA) in study 1 as having the highest average importance among the metabolites tested (FIG. 11). HPA was present at lower concentrations in Alzheimer's patients relative to the control samples. Without being limiting, HPA may disrupt normal brain function by inhibiting the activity of certain enzymes involved in neurotransmitter metabolism, leading to a decrease in neurotransmitter levels. This, in turn, can contribute to the decline in cognitive function seen in Alzheimer's disease. In addition, HPA may contribute to oxidative stress and inflammation in the brain, which could further exacerbate the damage to brain cells and contribute to the progression of Alzheimer's disease.

The inventors' model also identified trigonelline as having a high average importance among the metabolites tested (FIG. 11). Without being limited by theory, trigonelline has anti-inflammatory and antioxidant properties, which may help to protect brain cells from damage and reduce oxidative stress in the brain. The inventors observed that the model identified low levels of Trigonelline in the Alzheimer's disease patient samples.

C18:2 and C6:1 are types of fatty acid metabolites that have been implicated in the development of Alzheimer's disease. Studies have shown that individuals with Alzheimer's disease tend to have higher levels of C18:2 in their blood compared to healthy individuals. Some studies have suggested that individuals with Alzheimer's disease tend to have altered levels of C6:1 in their blood compared to healthy individuals. C18:2 fatty acid metabolite has been shown to contribute to the formation of beta-amyloid, a protein that forms the plaques that are a hallmark of Alzheimer's disease. Additionally, C18:2 has been shown to promote inflammation in the brain, which can contribute to the death of neurons and the decline in cognitive function seen in Alzheimer's disease. The inventors' model has identified lower levels of C18:2 and C6:1 in Alzheimer's disease samples, which is a surprising observation.

Homocysteine is an amino acid that has been implicated in the development of Alzheimer's disease. Elevated levels of homocysteine have been observed in individuals with Alzheimer's disease, and high homocysteine levels have been associated with an increased risk for the development of the disease. Homocysteine has been shown to disrupt normal brain function by inhibiting the activity of enzymes involved in neurotransmitter metabolism, leading to a decrease in neurotransmitter levels and a decline in cognitive function. The inventors' model has identified lower levels of homocysteine in Alzheimer's disease samples, which is a surprising observation.

### Example 2: Metabolomics Study 2

A second metabolomic study was conducted to identify the metabolites most implicated in Alzheimer's disease. In this study, there were 148 metabolites included in the analysis. A total of 64 samples were analyzed, 32 of which were Alzheimer's disease patients and 32 controls. No cases were excluded and log transformation and paretoscaling were used for normalization.

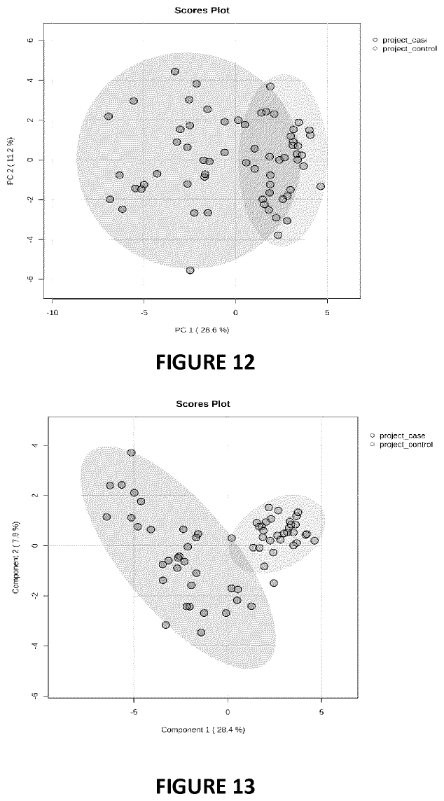

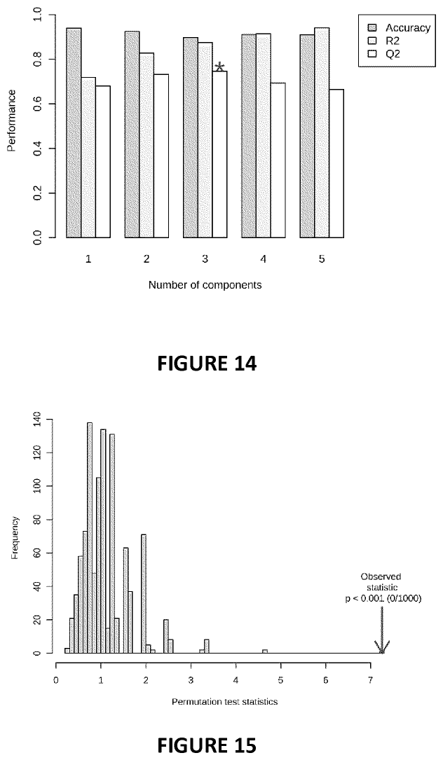

For the metabolites measured, there was a clear separation between Alzheimer's disease and control samples as shown FIGS. 12-15.

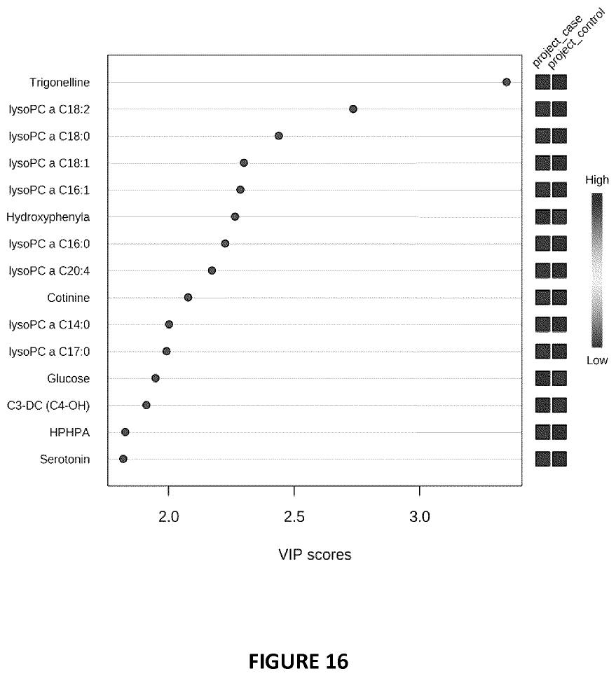

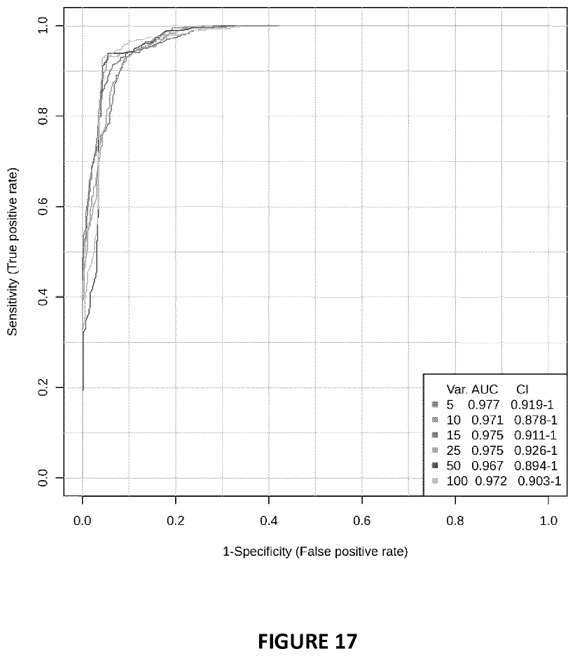

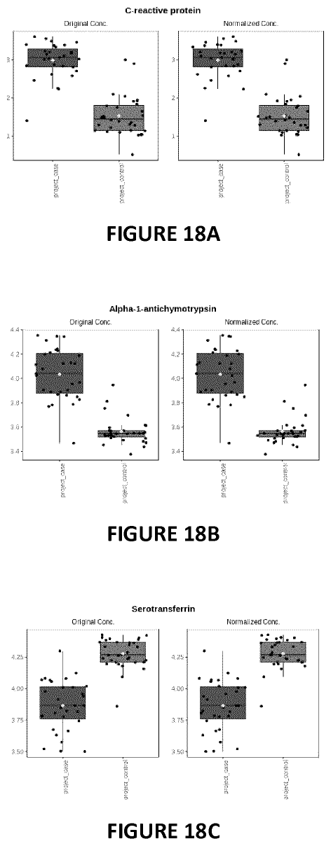

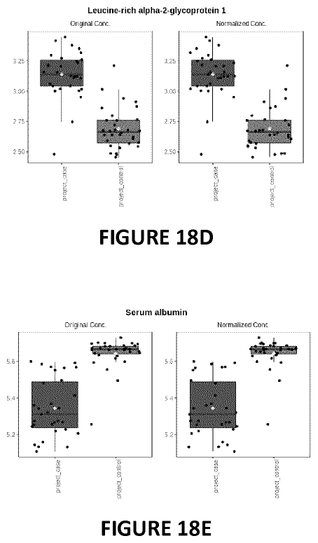

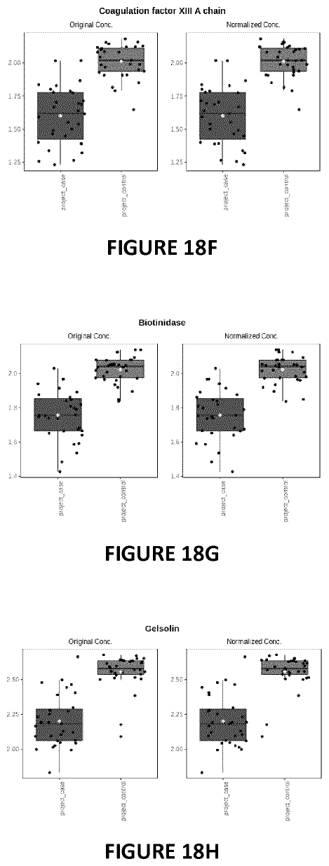

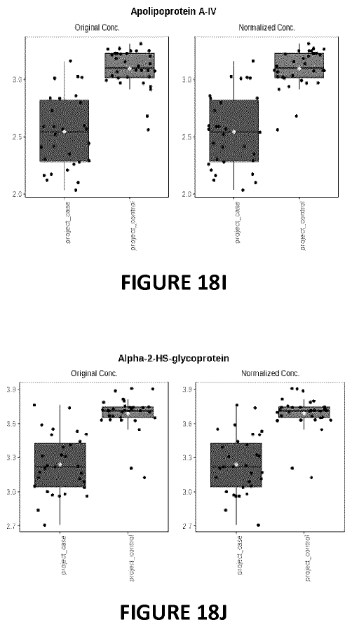

The top 15 metabolites ranked from study 2 based on average importance in the SVM model (VIP scores) are shown in FIG. 16. In both study 1 and study 2, trigonelline, C18:2 lysophosphatidylcholine, hydroxyphenylacetic acid (HPA) and 3-(3-hydroxyphenyl)-3-hydroxypropionic acid (HPHPA) were identified as having high average importance in the VIP plots (see FIGS. 11 and 16). Trigonelline was lower in this study and HPA was higher relative to the non-AD control cohort. Lysophosphatidylcholine a C18:0, lysophosphatidylcholine a C18:1, lysophosphatidylcholine a C16:1, lysophosphatidylcholine a C16:0, lysophosphatidylcholine a C20:4, cotinine, lysophosphatidylcholine a C14:0, lysophosphatidylcholine a C17:0, glucose, malonylcarnitine (C3-DC)+3-Hydroxybutyrylcarnitine (C4-OH) (C3-DC (C4-OH)) and serotonin were additional proteins in the top 15 metabolites identified by the VIP scores of inventors' model.

The metabolites identified in study 2 as having high VIP scores are summarized in Table 3 below.

TABLE 3
Top 15 metabolites identified by machine
learning in metabolomic study 2

| | | Higher/Lower than |
|---|---|---|
| No. | Alzheimer's Metabolite | control samples |
| 1 | Trigonelline | Lower |
| 2 | Lysophosphatidylcholine a C18:2 | Lower |
| 3 | Lysophosphatidylcholine a C18:0 | Lower |
| 4 | Lysophosphatidylcholine a C18:1 | Lower |
| 5 | Lysophosphatidylcholine a C16:1 | Lower |
| 6 | Hydroxyphenylacetic acid (HPA) | Higher |
| 7 | Lysophosphatidylcholine a C16:0 | Lower |
| 8 | Lysophosphatidylcholine a C20:4 | Lower |
| 9 | Cotinine | Lower |
| 10 | Lysophosphatidylcholine a C14:0 | Lower |
| 11 | Lysophosphatidylcholine a C17:0 | Lower |
| 12 | Glucose | Higher |
| 13 | Malonylcarnitine (C3-DC)+3- | Higher |
| | Hydroxybutyrylcarnitine (C4-OH) | |
| | (C3-DC(C4-OH)) | |
| 14 | 3-(3-hydroxyphenyl)-3- | Lower |
| | hydroxypropionic acid (HPHPA) | |
| 15 | Serotonin | Lower |

### Example 3: Proteomics Study 1

### Means Comparison

Of the 142 proteins included in the analysis, 87 biomarkers showed a significant difference between the case and control groups using the two-sample t-test for means comparison. The false-discovery rate was accounted and adjusted for.

Table 4 summarizes the 10 proteins with the lowest p-values:

TABLE 4
Proteins with the lowest p-values in study 1

| Biomarker | P-Value | FIG. |
|---|---|---|
| C-reactive protein | 1.9948E−17 | 18A |
| Alpha-1-antichymotrypsin | 2.9036E−16 | 18B |
| Serotransferrin | 1.599E−14 | 18C |
| Leucine-rich alpha-2-glycoprotein 1 | 3.8518E−14 | 18D |
| Serum albumin | 1.2482E−13 | 18E |
| Coagulation factor XIII A chain | 3.2921E−13 | 18F |
| Biotinidase | 6.2492E−13 | 18G |
| Gelsolin | 1.3695E−12 | 18H |
| Apolipoprotein A-IV | 3.2691E−12 | 18I |
| Alpha-2-HS-glycoprotein | 1.2782E−11 | 18J |

Leucine-rich alpha-2-glycoprotein 1 (LRG1) is produced in various tissues throughout the body, including the brain, liver, lung, spleen, and blood vessels. Leucine-rich alpha-2-glycoprotein 1 (LRG1) is a protein that has been implicated in various diseases. The inventors observed significantly high levels of LRG1 in the blood of the AD samples. This is likely being produced in response to AD disease mechanisms.

Serotransferrin, also known as transferrin, is a protein involved in iron transport and regulation in the body. Principal Component Analysis (PCA)

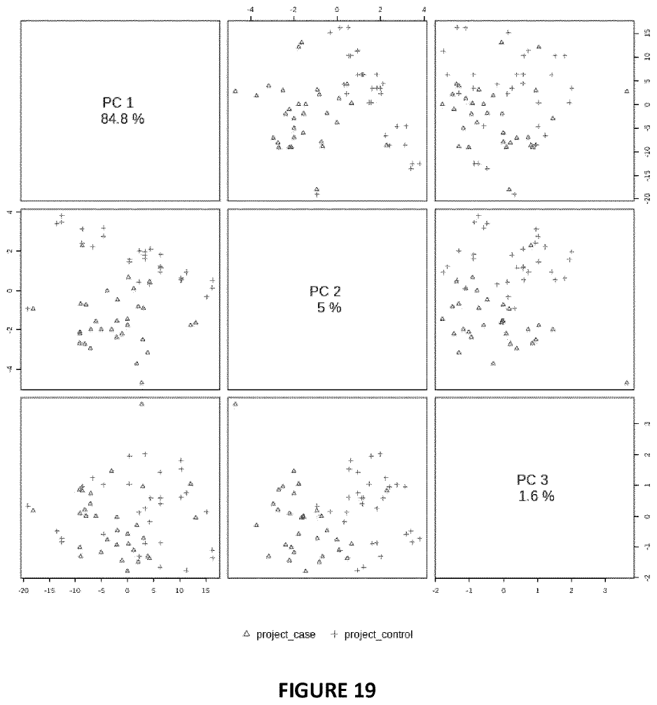

FIG. 19 shows pairwise scores plot for the top 3 PCs. PC1 explains 84.8% of the variation in the data. PC 2 and PC 3 account for 5% and 1.6%, respectively.

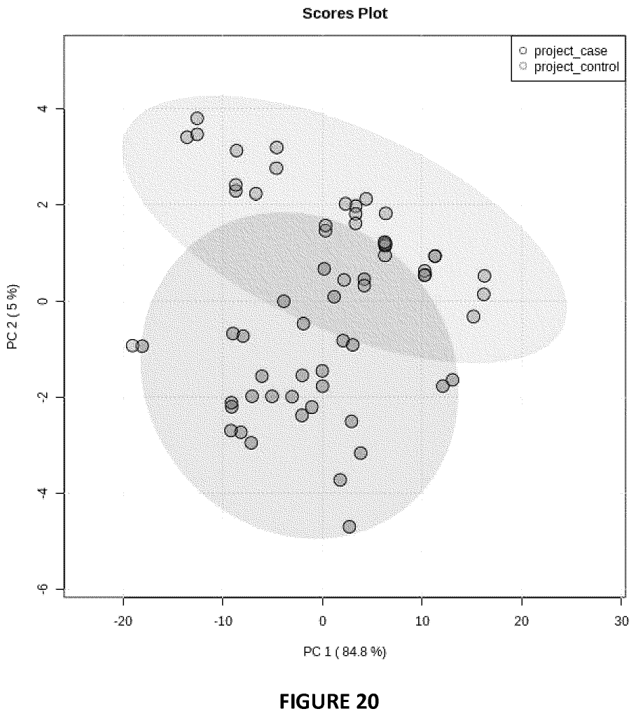

Similar to metabolomics, there appears to be a separation between biomarkers in the AD-case group and the control group, suggesting that a new biosignature can be identified for this disease (FIG. 20).

### Partial Least Squares Discriminant Analysis (PLS-DA)

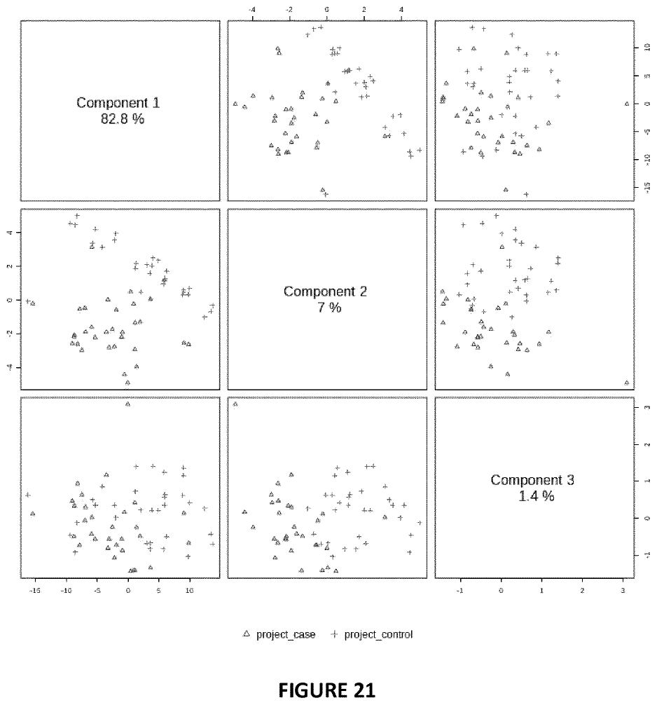

FIG. 21 displays the pairwise score plot for the top 3 components. Component 1 explains 82.8% of the variance in the data, component two explains 7%, and component 3 explains 1.4%.

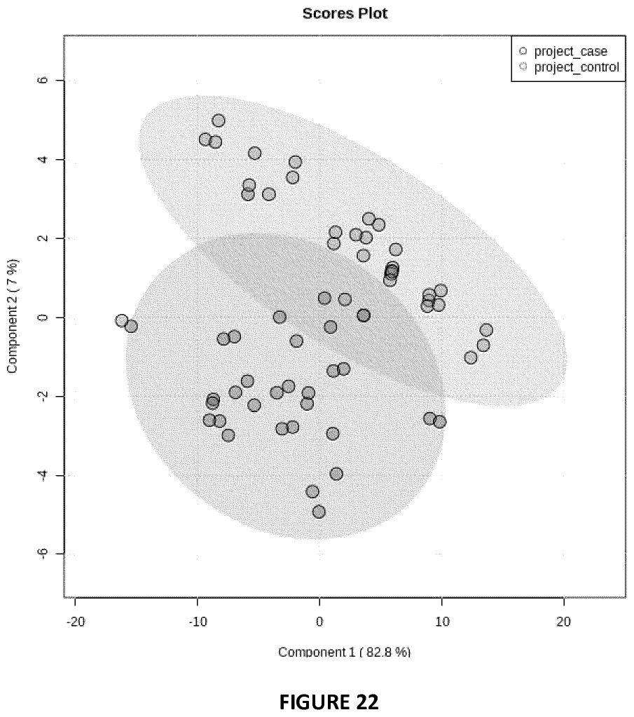

Similar to the PCA analysis, there is separation in proteins using PLS-DA (FIG. 22). In general, this shows that the PLS-DA model has a good ability to classify the samples into the two groups based on the predictor proteins used.

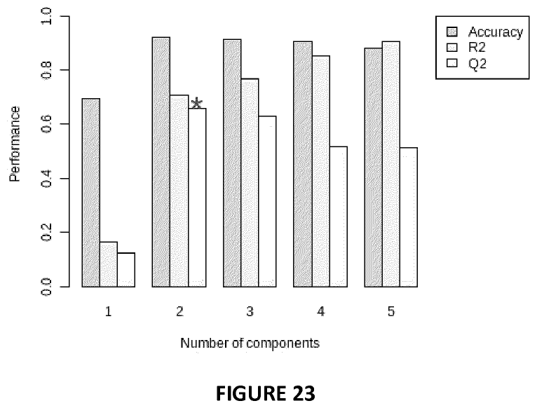

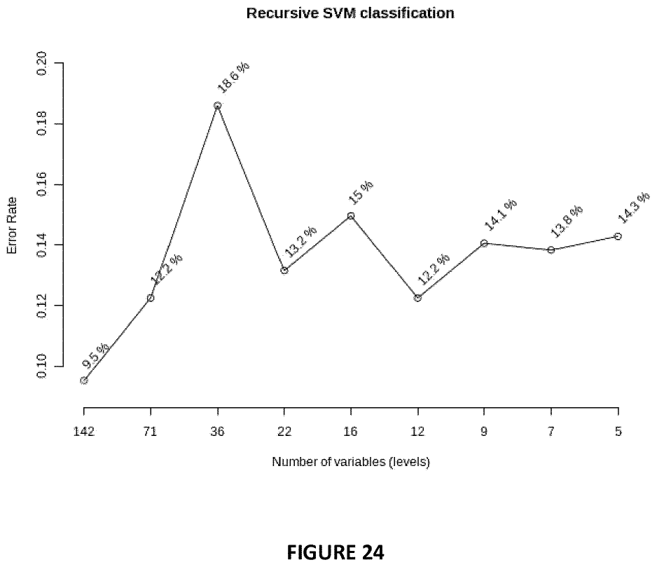

The performance of each component was assessed using the accuracy, R2, and Q2. The results are in FIG. 23. The Recursive SVM classification is shown in FIG. 24.

### Linear SVM

Linear SVM was used as the classification method for proteomics data. This model using proteomics data has an average predictive accuracy of 88.4%.

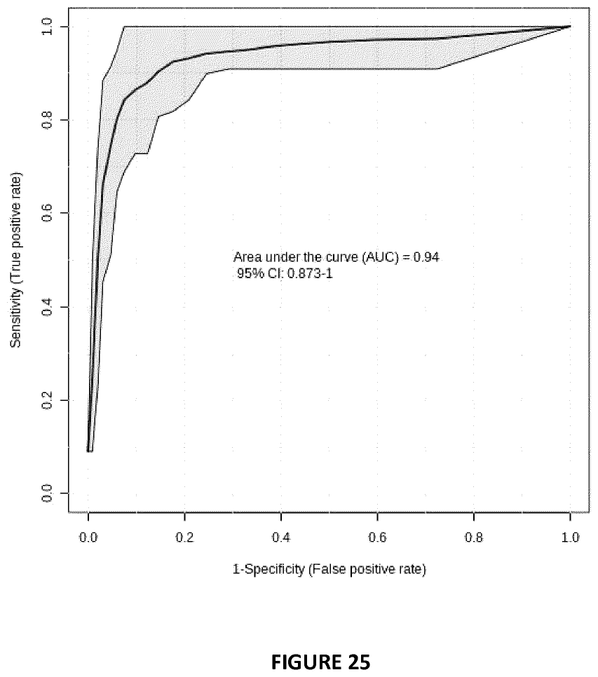

FIG. 25 shows the Receiver Operating Curve (ROC) for the SVM model along with the 95% confidence interval. The area under the curve (AUC) is 94.0%.

When separating Alzheimer's disease (positive class) from healthy (negative class) individuals, the model is able to, on average, correctly classify new data points to the correct class 88.4% of the time. In addition, an AUC of 94.0% would indicate that the model is able to correctly classify individuals with Alzheimer's disease as positive and healthy individuals as negative in 94.0% of the cases. This means that the model is able to correctly separate the two classes in most cases, and thus it would be considered to have good performance.

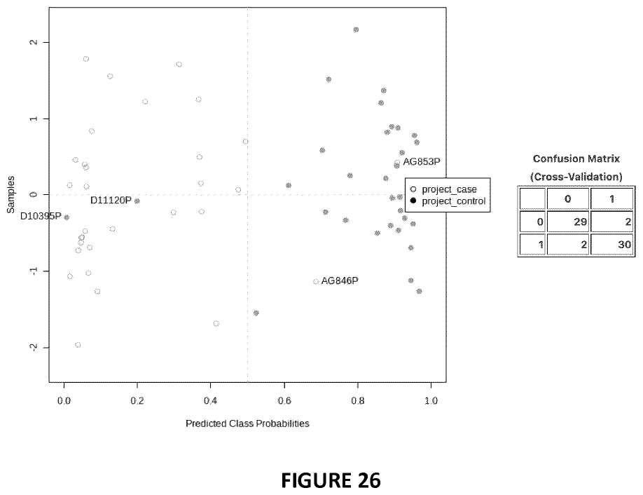

FIG. 26 shows the predicted classification of the data set using linear SVM modeling. In this iteration, there were 2 false positives and 2 false negatives out of 63 samples.

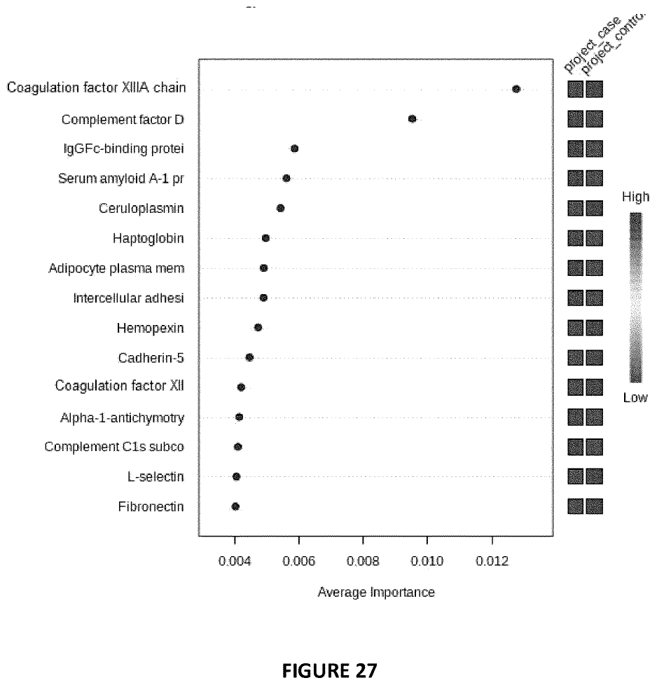

FIG. 27 shows the top 15 proteins ranked based on average importance in the SVM model.

The proteins identified by the SVM model as having high predictive ability are summarized in Table 5 below.

TABLE 5
Top 15 proteins identified by machine learning in study 1

| | | Higher/Lower |
|---|---|---|
| No. | Alzheimer's Protein | than control samples |
| 1 | Coagulation factor XIII A Chain | Higher |
| 2 | Complement factor D | Lower |
| 3 | IgGFc-binding protein | Lower |
| 4 | Serum amyloid A-1 protein | Lower |
| 5 | Ceruloplasmin | Higher |
| 6 | Haptoglobin | Lower |
| 7 | Adipocyte plasma membrane | Lower |
| 8 | Intercellular adhesion protein | Lower |
| 9 | Hemopexin | Higher |
| 10 | Cadherin-5 | Higher |
| 11 | Coagulation factor XII | Higher |
| 12 | Alpha-1-antichymotrypsin | Lower |
| 13 | Complement Cls subcomponent | Lower |
| 14 | L-selectin | Higher |
| 15 | Fibronectin | Higher |

Coagulation Factor XIII (FXIII) is a protein involved in the process of blood clotting.

Serum Amyloid A1 protein (SAA1) is a protein that is primarily produced by the liver and is involved in the body's response to inflammation.

Alpha 1 Antichymotrypsin (A1AT) is a protein that is involved in the regulation of inflammation and the clearance of proteases, which are enzymes that break down proteins.

The role of Adipocyte plasma membrane-associated protein (APMAP) in Alzheimer's disease is not well understood and has not been extensively studied. Currently, there is limited evidence to suggest that APMAP may play a role in the development of Alzheimer's disease.

### Example 4: Proteomics Study 2

A second proteomic study was conducted on the same patient samples as metabolomic study 2 (Example 2). In the second study, there were 140 proteins included in the analysis. A total of 64 samples were analyzed, 32 of which were Alzheimer's disease patients and 32 controls. Two cases were excluded and log transformation and paretoscaling were used for normalization.

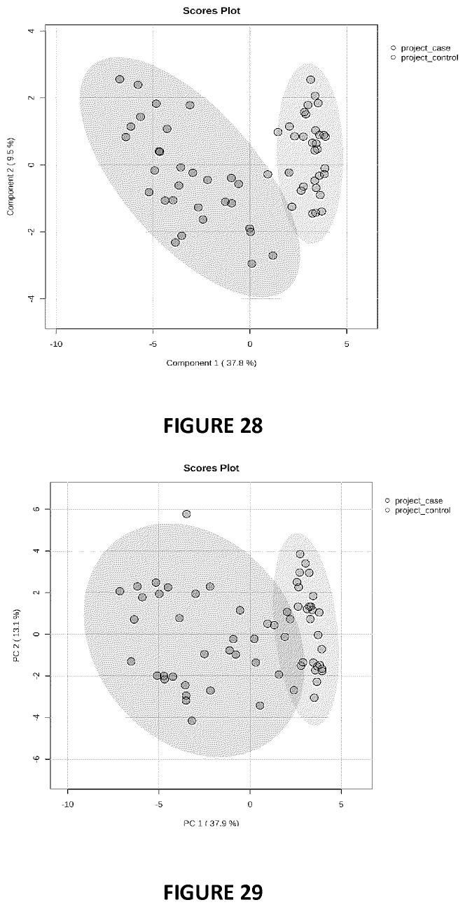

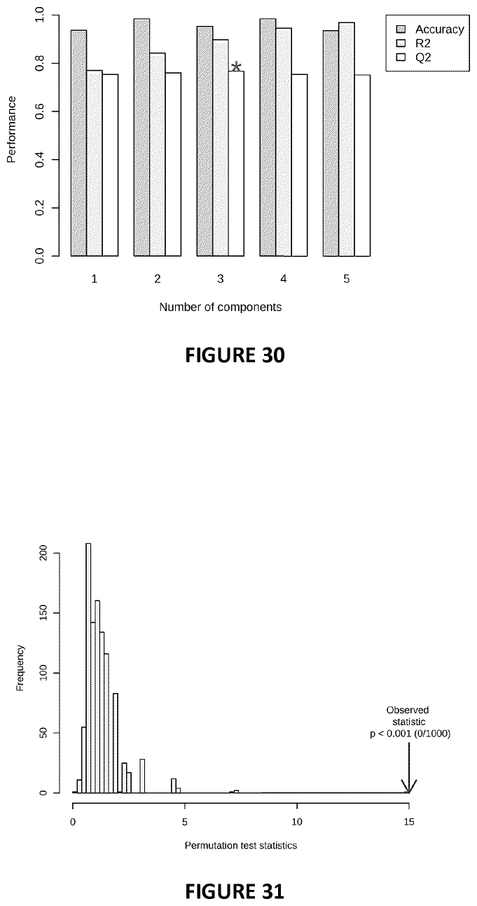

There was a clear separation between Alzheimer's disease and control samples as shown FIGS. 28-31.

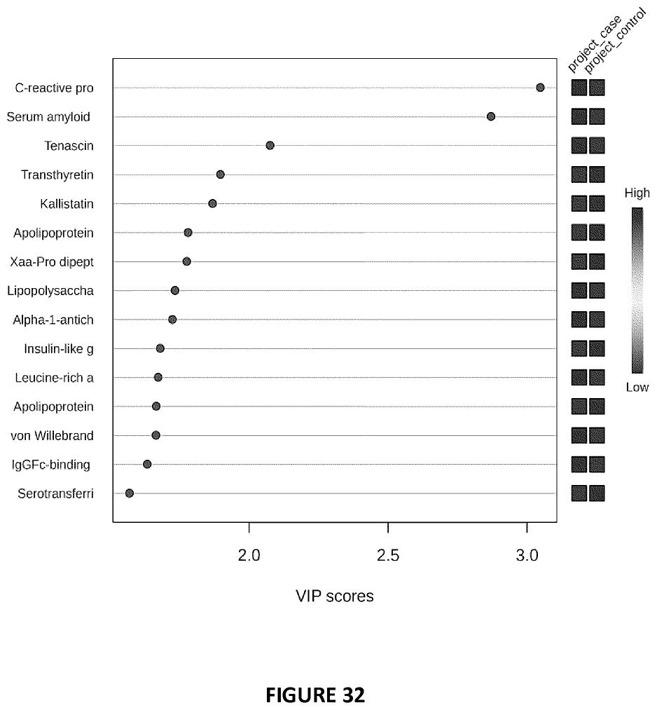

The top 15 proteins ranked based on average importance in the SVM model are shown in FIG. 32. The proteins are summarized in Table 6 below.

In both study 1 and study 2, serum amyloid A-1 and alpha-1-antichymotrypsin were identified as having high average importance in the VIP plots (see FIGS. 27 and 32). Serum amyloid A-1 proteins and alpha-1-antichymotrypsin levels were higher relative to the non-AD control cohort. C-reactive protein (CRP), tenascin-C, transthyretin, kallistatin, apolipoprotein A-IV, Xaa-Pro dipeptide, lipopolysaccharide-binding protein, insulin-like growth factor, leucine-rich alpha-2-glycoprotein, apolipoprotein, Von Willebrand, IgGFc-binding and serotransferrin were also identified in the top 15 proteins.

TABLE 6
Top 15 proteins identified by machine learning in proteomic study 2

| | | Higher/Lower than |
|---|---|---|
| No. | Alzheimer's Protein | control samples |
| 1 | C-reactive protein (CRP) | Higher |
| 2 | Serum amyloid A-1 | Higher |
| 3 | Tenascin-C | Higher |
| 4 | Transthyretin | Lower |
| 5 | Kallistatin | Lower |
| 6 | Apolipoprotein A-IV | Lower |
| 7 | Xaa-Pro dipeptide | Lower |
| 8 | Lipopolysaccharide-binding protein | Higher |
| 9 | Alpha-1-antichymotrypsin | Higher |
| 10 | Insulin-like g | Lower |
| 11 | Leucine-rich alpha-2-glycoprotein | Higher |
| 12 | Apolipoprotein | Lower |
| 13 | Von Willebrand | Higher |
| 14 | IgGFc-binding | Higher |
| 15 | Serotransferrin | Lower |

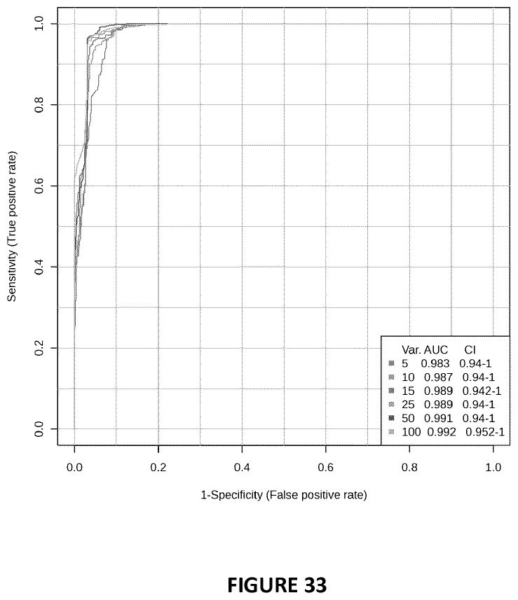

A ROC curve for the SVM model with the 95% confidence interval is shown in FIG. 33. The ROC curve demonstrates the high diagnostic ability of the inventors' model.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

Headings used throughout the specification should not be construed as limiting to the invention.

While particular embodiments of the present disclosure have been illustrated and described, it would be obvious to those skilled in the art that various other changes and modifications can be made without departing from the scope of the present disclosure. It is therefore intended to cover in the appended claims all such changes and modifications that are within the scope of this disclosure.

1. A method for diagnosing and treating Alzheimer's disease in a subject, the method comprising:
(a) providing a biological sample obtained from the subject;
(b) measuring concentration levels from the obtain sample, at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0 or C18:2 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin; and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor;
(c) comparing the concentration levels of the Alzheimer's-related metabolites and/or Alzheimer's-related proteins from the obtained sample to the concentration levels of corresponding reference Alzheimer's-related metabolites and/or Alzheimer's-related proteins from an Alzheimer's-negative sample;
(d) identifying the subject as having Alzheimer's disease if the concentration levels of the Alzheimer's-related metabolites and/or Alzheimer's-related proteins from the obtained sample are different relative to the concentration levels of the reference Alzheimer's-related metabolites and/or Alzheimer's-related proteins from the Alzheimer's-negative sample; and
(e) treating the subject so identified with an Alzheimer's treatment regime by administering an agent effective to treat, ameliorate or reduce the symptoms Alzheimer's disease, wherein the treating comprises adjusting the levels of one or more of the metabolites and/or proteins identified as being present at levels different than the control.

2. The method of claim 1, wherein the Alzheimer's-related metabolites are selected at from least Hydroxyphenylacetic acid (HPA); Trigonelline; Lysophosphatidylcholine (lysoPC); C18:2 fatty acid metabolite; C6:1 fatty acid metabolite; and/or at least Homocysteine; and the Alzheimer's-related proteins are selected from Coagulation factor XIII A Chain; Complement factor D; IgGFc-binding protein; Serum amyloid A-1 protein; Ceruloplasmin; Haptoglobin; C-reactive protein and/or Tenascin C.

3. The method of claim 2, wherein the Alzheimer's-related metabolites are selected from at least Hydroxyphenylacetic acid; or Trigonelline; and the Alzheimer's-related proteins are selected from at least Coagulation factor XIII A Chain; Complement factor D; or C-reactive protein.

4. (canceled)

5. (canceled)

6. The method of claim 3, wherein the Alzheimer's-related metabolites are selected from at least Hydroxyphenylacetic acid (HPA); or Trigonelline; and the Alzheimer's-related proteins are selected from Coagulation factor XIII A Chain or Complement factor D.

7. The method according to claim 1, wherein the Alzheimer's treating comprises adjusting the blood levels of one or more of the Alzheimer's-related metabolites and/or the Alzheimer's-related proteins in the subject diagnosed as having the Alzheimer's or predisposed of developing the Alzheimer's or a combination thereof.

8. The method according to claim 7, wherein the adjustment of the blood levels of one or more of the Alzheimer's-related metabolites and/or the Alzheimer's-related proteins in the subject occurs until an improvement in the Alzheimer's disease symptoms in the subject is observed.

9. The method according to claim 8, wherein the adjustment of the blood levels of one or more of the Alzheimer's-related metabolites comprises adjusting the composition of gut microbiota in the subject.

10. The method according to claim 1, wherein the identifying step (d) occurs upon determination that the concentration levels of at least one, at least two, at least three, at least four or at least five of the Alzheimer's-related metabolites and/or the Alzheimer's-related proteins from the obtained sample differ by about 20% or more, about 30% or more, about 40% or more, about 50% or more, about 60% or more, or about 70% or more relative to the concentration levels of the reference Alzheimer's-related metabolites and/or Alzheimer's-related proteins from the Alzheimer's-negative sample.

11. The method according to claim 1, wherein the identifying step (d) occurs upon determination that the concentration levels of the Homocysteine, C18:2 fatty acid metabolite and/or C6:1 fatty acid metabolite from the obtained sample are decreased relative to the concentration levels of the corresponding reference Alzheimer's-related metabolites and/or Alzheimer's-related proteins from the Alzheimer's-negative sample.

12. The method according to claim 1, wherein the identifying step (d) occurs upon determination that the concentration level of Coagulation factor XIII A Chain and/or Hydroxyphenylacetic acid (HPA) from the obtained sample are increased relative to the concentration levels of the corresponding reference Alzheimer's-related metabolites and/or Alzheimer's-related proteins from the Alzheimer's-negative sample.

13. (canceled)

14. (canceled)

15. (canceled)

16. (canceled)

17. The method of claim 1 further comprising monitoring the subject for progression or regression of Alzheimer's disease, the monitoring comprising:
providing a second obtained biological sample obtained from the subject at a second time point that is after a first time, the first time being the step (a) of providing a biological sample obtained from the subject;
assessing a second Alzheimer's-related metabolite and/or Alzheimer's-related proteomic profile by measuring concentration levels of the Alzheimer's-related metabolites and/or Alzheimer's-related proteins from the second obtained biological sample;
comparing the second Alzheimer's-related metabolite and/or Alzheimer's-related proteomic profile with the reference Alzheimer's-related metabolite and/or proteomic profile from the Alzheimer's-negative sample;
determining that there is a second difference between the first Alzheimer's-related metabolite and/or proteomic profile and the reference Alzheimer's-related metabolite and/or proteomic profile from the Alzheimer's-negative sample, the second difference being indicative of Alzheimer's; and
determining Alzheimer's progression based on at least in part on the first and second differences
treating the subject as identified with an Alzheimer's treatment by administering an agent after the second time point in a second step of treating, the second treating comprising adjusting the levels of one or more of the metabolites and/or proteins identified as being present at levels different than the control.

18. (canceled)

19. (canceled)

20. (canceled)

21. (canceled)

22. (canceled)

23. A computer-implemented method for processing a biological sample of a subject, diagnosing Alzheimer's disease and optionally causing treating of the Alzheimer's, the computer-implemented method comprising:
(a) receiving a biological sample obtained from the subject;
(b) processing the sample in a spectroscopy unit directly or wirelessly linked to a processing device, the processing device having memory for storing measurement data from the spectroscopy unit;
(c) in the spectroscopy unit, measuring levels of at least one, at least two, at least three, at least four or at least five Alzheimer's-related metabolites selected from Hydroxyphenylacetic acid (HPA); Trigonelline; a Lysophosphatidylcholine (lysoPC) selected from a C18:2, C18:0, C18:1, C16:0, C14:0, C16:1, C20:4, C17:0; C26:0 fatty acid metabolite; C6:1 fatty acid metabolite; Homocysteine; Putrescine; C14:1-OH; Histidine; LysoPC a C26:0; Alanine; Pyruvic acid; 3-(3-hydroxyphenyl)-3-hydroxypropionic acid (HPHPA); Trimethylamine N-oxide (TMAO); Benzoic acid; Citrulline; Cotinine; Glucose; C3-DC)+3-Hydroxybutyrylcarnitine (C4-OH) (C3-DC (C4-OH)); and/or Serotonin and/or at least one, at least two, at least three, at least four or at least five Alzheimer's-related proteins selected from C-reactive protein; Alpha-1-antichymotrypsin; Serotransferrin; Leucine-rich alpha-2-glycoprotein 1; Serum albumin; Coagulation factor XIII A chain; Biotinidase; Gelsolin; Apolipoprotein A-IV; Alpha-2-HS-glycoprotein; Complement factor D; IgGFc-binding protein; Serum amyloid A-1 protein; Ceruloplasmin; Haptoglobin; Adipocyte plasma membrane; Intercellular adhesion protein; Hemopexin; Cadherin-5; Complement C1s subcomponent; L-selectin; Fibronectin; Tenascin-C; Transthyretin; Kallistatin; Xaa-Pro dipeptide; Lipopolysaccharide-binding protein; Insulin-like growth factor; and/or Von Willebrand factor and storing the measurement data in the processor; or (d) comparing the stored measurement data to a value in the memory representing an Alzheimer's-negative sample using multivariate statistical analysis; and
(e) storing on the processing device a result corresponding to at least one, at least two, at least three, at least four or at least five Alzheimer's-related metabolites and/or Alzheimer's-related proteins from the obtained sample, wherein the result identifies the subject as having Alzheimer's if the measurement data representing the levels of the Alzheimer's-related metabolites and/or Alzheimer's-related proteins are different relative to a concentration levels of reference Alzheimer's-related metabolites and/or Alzheimer's-related proteins from an Alzheimer's-negative sample;
(f) displaying an Alzheimer's treatment regime on an electronic display connected directly or wirelessly to the processor for the subject identified as having Alzheimer's or as having predisposition of developing Alzheimer's, the displayed treatment regime comprising electronic text on a graphical user interface; and
(iv) adjusting blood levels of one or more of the Alzheimer's-related metabolites and/or Alzheimer's-related proteins in the subject diagnosed as having or predisposed of developing the Alzheimer's by administering an agent effective to adjust the blood levels of the metabolites until an improvement in the cognitive performance in the subject is observed.

24. The method of claim 23, wherein the Alzheimer's-related metabolites are selected from Hydroxyphenylacetic acid (HPA); Trigonelline; Lysophosphatidyl choline; C18:2 fatty acid metabolite; C6:1 fatty acid metabolite; and/or at least Homocysteine; and the proteins are selected from at least Coagulation factor XIII A Chain; Complement factor D; IgGFc-binding protein; Serum amyloid A-1 protein; Ceruloplasmin; C-reactive protein, Tenascine C and/or Haptoglobin.

25. The method of claim 24, wherein the Alzheimer's-related metabolites are selected from at least Hydroxyphenylacetic acid (HPA); or Trigonelline; and the Alzheimer's-related proteins are selected from at least Coagulation factor XIII A Chain or Complement factor D.

26. (canceled)

27. (canceled)

28. (canceled)

29. The method of claim 1, further comprising adjusting or causing the adjustment of the levels of the metabolites and/or proteins comprises nucleic acid therapy, wherein the nucleic acid therapy comprises reducing the level of a protein expressed or metabolite produced by siRNA or antisense therapy, or wherein the nucleic acid therapy comprises increasing the level of a protein expressed or metabolite produced by mRNA therapy.

30. (canceled)

31. (canceled)

32. (canceled)

33. The method of claim 23, wherein the metabolite and/or proteome profile has been previously identified as having the predictive value by classifying disease and non-disease samples into the two groups by a computer-implemented method based on the predicted metabolites and/or proteins used in the computer model and assessing whether the groups are separated.

34. (canceled)

35. The method of claim 1, wherein the proteins or fragments thereof and/or metabolites are at least one, two or all of Homocysteine, C18:2 fatty acid metabolite and C6:1 fatty acid metabolite and wherein measured levels of the corresponding at least one, two or all of Homocysteine, C18:2 fatty acid metabolite and C6:1 fatty acid metabolite are reduced relative to an Alzheimer's-negative sample.

36. The method of claim 35, wherein the proteins, fragments thereof and/or metabolites are at least Homocysteine and C18:2 fatty acid metabolite.

37. The method of claim 35, wherein the proteins, fragments thereof and/or metabolites are at least Homocysteine and C6:1 fatty acid metabolite or wherein the proteins, fragments thereof and/or metabolites are at least C18:2 fatty acid metabolite and C6:1 fatty acid metabolite.

38. (canceled)

39. The method of claim 37, further comprising measuring metabolites selected from at least one of Hydroxyphenylacetic acid (HPA); Trigonelline; Lysophosphatidylcholine (lysoPC); C18:2 fatty acid metabolite; C6:1 fatty acid metabolite; and Homocysteine or further comprising measuring proteins selected from at least one of Coagulation factor XIII A Chain; Complement factor D; IgGFc-binding protein; Serum amyloid A-1 protein; Ceruloplasmin; Haptoglobin; C-reactive protein and Tenascin C.

40. (canceled)
